SEQUENCE LISTING (16 sequences)

SEQ ID NO: 1 - DNA, 228 bp, synthetic construct
  source 1..228: mol_type = other DNA
cgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 2 - DNA, 529 bp, synthetic construct
  source 1..529: mol_type = other DNA
gcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 3 - DNA, 27488 bp, synthetic construct
  source 1..27488: mol_type = other DNA
catcatcaataatatacaggacaaagaggtgtggcttaaatttgggtgttgcaaggggcggggtcatgggacggtcaggttcaggtcacgccctggtcagggtgttcccacgggaatgtccagtgacgtcaaaggcgtggttttacgacagggcgagttccgcggacttttggccggcgccccgggtttttgggcgtttattgattttgcggtttagcgggtggtgcttttaccactgtttgcggaagatttagttgtttatggagctggttttggtgccagttcctccacggctaatgtcaaagtttatgtcaatataacagaaacactctgttctctgtttacagcaccccacctagtcgactaaaaaacctcccacacctccccctgaacctgaaacataaaatgaatgcaattgcgcgatttaaatcgcgcggcgggtacctactttgtcaatgtgtgtaattgcagcccctcggggggttgtgcagagaatttatttgtttggtgtgtatgatgctacttgtgccattatgcaactgggcgaggcaggcaatgctgctagtgaaagactgtgtacttgcgggttcagacacagcaccccttccctgcgggccacctatgtaactgacaccaggattgaccgggagctgaactctcaagacacggctgagttctttagcagtgatgaagataatttttaggtgagtagatgggcgtggtttgggggagtataaaaggggcgcggtacgtggctgtgtatttacagccatggaccctcaacagaaggggcttgtgaacacgtgttttgtgactacgcgtattccgtcttgggcaggagcaagacagaatgtcaccgggtcagatttagaaggaaagcccgtgccctcagatgtgctggaaagtggacgcccgcttgcagccccgcgcatcagaactttgtatgaggagcagcagctgaacatgcttgcggtgaatgttcttttggatgagctgaagatccaggtggctgccatgcaaaactctgtgactgctattcagcgagaagtaaatgatctaaagcaacgaatcgcccgagattaatgtaaaaataaaatttatttcttttttgaatgataataccgtgtccagcgttgtctgtctgtaatagttctatgaattctttccatggcttggtacactttagcttgaatgttaaggtacatggggatgagtccctcgtcggggctcaagtacagccagtgcatacaattgtcaatgggctcagtattgtatacaatccaatcaaagttagtattgaggcggtggtagttaaaaatatccttaagcaggagggaaatagcagtgggttgtcccttagtgtagatgttaataaacctattaagttgggaaggatgcaccttaggactgatgatatgcatttttgcttggatttttaagttggcaatgtttcccccctggtctcgtctgggattcatgttgtgcaagaccactataacagagtagcctgtgcacttgggaaacctgtctagcagtttagagggaaaggcgtgaaagaacttggctatgcccctgttgccccctaagtcttccatgcactcatccattacaatagctagtggccctttagaagcggcccttgcaaaaacattggtagggttggtaacatcatagttggcctcattggtgagttctgcataagacatttgcacaaacttgggcatgatgctgccactctgaggaaccaccgtgttttcggggcctgcaaggaagtttccctcacatatttgggtttcccatgctgcaatttcctggggaggaatcatgtccacctggggggtgataaaaaagactgtttctggggcggggactataagctgggcagacatgaggttcctaagaagctgggacttgccacaccctgtgggcccatacactgcagcaataaagggttgcttgtagtagttaagggatgcacagcttccatctttattaaggtacttactgaccttcatgcaacactgggttatgctatcttgaatatctagtaggtcatttaacagactgtctccccccatagaaagcagttcctcaaaagaggcaaagtttttgaggggcttaagtccctctgcatatggcatgtttgttaagctttgctgaagagtttgcaacctgtcccagaggctttttaagtcatccactgcagctccatccaggtagtttctgtgtttcttgggtttggatgcttgcggctgtatggaatgagacgatgcatgtctagagcatgcagggttttgtccttccatggcctcagcttccttgttagggtggtttctgtcactgtgaaaggttgcacgtgggcctgcacgctggtgagggtcctgcgcaggctcatcctgctggttttgaagacgtccgagccctgctgcatgtcagcatagtagcaagcttttagcacatcataagagagggtggaggtggcatgacctttggccctgagcttgcccttgcccacgtgatggcacttagggcaggtggtgtccttgagagcatacaacttgggcgccaggtacactgattctgagctgtaagcatccgatccacacttctcacactgtgtttcacactctactagccaagtgagctctgggttgctgggatcaaagactagcttgcctccattttttttgagtcgtttcttacctttttcttccatcaatttgtaaccctccatggtggtgaagaggctgtcagtgtctccgtagacagattttaggaccctgttttcaagtggggttcccgcgtcgttttcgtagagaaactgagaccactctgagacaaatgcccgggtccatgctagaacaaaggaggcaatttgtgagggatatctgttattggctattaggggggtggacttttctaaagtgtgtaaacaaaagtcatcatcttcagcatcaataaacatgattggcttataatggtatgacgtcatgctaaaattatgcataaaaggcgcttcggcgagggggtctttagttggatcttcttcgctgcaagaaggcggtgccaactggcgaacgtcagaattgacaggtgggtacgctacgacaaattctggcatgatttctgcacacaaattgtctgtttctatgtatgaggatgatttgactacataggcaccagaagcaatttctttagcaatgtttgactctatttgatctgaaaaaactgtttttttgttgtctagcttggtagcaaaagacccatagagggcatttgacagcagcttagcaatgctgcgcatggtttgatttttgcttttgtctgcttcttctttggcactgatgttgagctgcacatactctctggcgagacatttccactctggaaacacggtggttctggtatcagggatgagggtaaccttccacccgcggttgtgcagggtaataacatctatggaggttgcaatctccccgcgtaggggctcgttggtccagcaaagcctgccccccttgcgtgagcaaaatgggggcagcacgtccaaaaactcttctgctggtgggtcagcatcaatggtgaagatgcccggaagaaggtccttatcaaagtagctaatagttttgtgatttagcattttttgctcataagcctttatggccagcgcccgctcataggggtttagcggtgaaccggcgggaaaagggtgtgtaagggcacttgcatacatgccacatatgtcatagacaaacactggctcttgcaacactccaatgtaagtggggtagcacctgcctcctctgatgctaagccttacatactcatacatttcattggagggcgccatgaggatggtacttaggtggtgtgtgttaggcttttctgctttatacaagatttgtttaaaaatggcatgggagttggagctgatggttggacgctggaagacgttaaaggaggtttcaggtaagtttacagagtttttaatgaagatttggtaggattctataagtttgtttaccaactgggctgttacttgaacgtcgagtgcgcagtaattcaagggtttgctgaattatgtcgtaagcgccctttttttcttttatccaaaggtctttgttaaaggagtactcctcctgatccttccagtactttaagtccggaaagccatcggcgtcttgttggtaagagccaagcatgtaaaactcattcacagcctggtagggacagcagcctttggaaaccgttaggctgtatgctttggcagcatttttgaggcttgtgtgtgttaaagagaaagtgtcacgcaccataaattttatgtactggcttttcaagtcagacgccagtacttggccctgctcccagtgctcgtaactcttggcaggcacgtagctggggttaggcagagagtaagtaatatcattgaacagcaaccttccagcccgcggcatgaagttgcggcaaatttcaaacacgggtggtatttctgcccgcgtgctaaccacctgagctgctagcaatatttcatcaaaccccgtgatgttgtgtcccactgcatacacctcaataaatcttgggtttcctttgagcttgaatttttttaactgatctggagtaagctcttcagggctttctagtcccaaagtagtggctttttcttgaagctggggattttggcaaatcacgtgggaccagaggctggcggcaatatgctgctgcaaggtgtctctgaactttttgaatttaactcctatggctcttttttctggagtgatgcaatagtacagagtgtcctgctctttaccattccaaatttcccagttttgctctgttgcaagagttttggcaacattgaccaggtggtcgtcccccagcagttggaaaactagcataaatggcacaagttgctttccaaacttgccgtgccacgtgtacgtttccacatcatatacaataaacagcctttctgtgttgggcggggccccaatgggggtgaaagatattttttcccaccagtctgcgctgtgtgcggccacgtgatggaagtaaaagtcccgtcttctgagggcgcaggtgtgagtaactttaaaaaaagagccgcagaactcacacttttgcatttgagagatgtcttttattaagaagactttaccttgcttaactaaaaacttgagtgggaagctgagcagcggaacctccgcttgaaactctaccctcctcactgttcctcgctggtagtagcaaattagactgtagggtgccgcggcctcctctatgagggcgggcaggttgtgggcagtgatgggcctgtttaagcatgcgggtaggtcatagaggttagtgttgcaaaggttagtaagcgcgctggccaagttggagtgatacttaatttccacacactgtccattatccaacacggcggaaatgcttagtgtggcgcgctggaccactacggcgcccttgtacttgcgcttgggtgggtttactatggtggaagttgcacctgctcgtggtgagctggcaaaggcctgtgctgcatcctgagctgggaggctagggccactactctcctgttgattgattggatttggcggttctgtgaaaagaccactggtcccgtgaccttgaacctaaaagaaagttccatagagtcaacctctgtatcattaatggccacttgtctgagaatttcgctcacgtccccgctgttttcatgataagcaatatctgccataaactgctccatttcctcctcctctagctcccctagcccagccctttctacggtggcagccaggtctcccgacacgcgggccataacctggctaaaggcattttctcccatttcattccacacccgactgtagaccacctgcccctcgtggtccctagcccgcatgaccacttgagctagctctaggtcaacccagcgggagcagggtgggtacagcctaaggttgtggtgcaggtagttgagggtggtggctacgtgctcagctacaaaaaaatacatgacccaacggcggatggtgagctcattaatgttgcccagcatttccaggcgctgaatgacctcataaaagtcaactgcaaagttaaaaaactgttcattgcgagcagacactgtgagttcctcttgcaggaggcggatagcttctactatagtgtctcggacttcttcttcaaaggaacggcgaggaggggaggcaggaggaggaggagaggcctctgcttcgggctcagacagctcctcagacaccggaggcggcgggggagccctgcgtctgcgcctgcgcactggaagcctgtctataaacctttctatcacctctccccgcctgcgcctcatttcctctgtaactgcacgtccattttccctgggcctaagctcgaaggctcccccagagagcggggtgcagggcgcgggtctgggcaggctcagggcgctaattatgctttttgttacagattggctgggtaaagactgaattgtttgaaactgtgacagctccggatcagtgaatttttccacaaaggcatcaagccacaaacaatcacaaggtaagcttagcatggtgtgcgggttgcgggggctaactaaaaagttgaaaaaagctgtcttaagcttacggatggtggttagaataactaggtctttgttgcccgcctgctggatgcgcatgcggtctgccagtccccatgcttcgttttgacactgggacaggtttttgtaatggtcttgcaagagcccctctacgggcacctgcctgtcgcctgccatgttggtggtgccatagcccctaagtggcctaataagggccaaatcggccactacgcgctctgccaggaccgcttgctgaacctgcgccagggtctctgcaaagttttcaaagtcaatgaacctgtggtaagctccagtgttaatggtgtatgagcagttggccatcaccgtccagtttagtttttgctgcaagggcaggtgtatctctgtgtactttagcctgctgtaggacctggtgtcaaagacgtagtcattgcacgttctcactaggtactggtagcccaccaaaaggtggggcggggggtaccaaaacagtggccagccctcggtagcgggctctcgagggctgaggttcataagcatgaggcggtggtattggtatatgtacttggacatccatgccagccctacagaggtggtggaagctctggtgtactctcgaacccggttaaaaatgtttctgagaggcctgaacacctcaacggtgtatggggtttgtccggttaggcgggcgcagtccagcgcgttctgcaaaacaaacaaggagtcgatggcactttcattcgctccattgcagatgcatccggtactgcgacagatgaagcccctgcccgcctctgccagcaagctagaagcagagagcgagggtttggcccgcttacaagggggtgctgctccagagttgcacccccgggtgcagatgaagcaagaggcggcggaggcttacatccccactagcaatgtgtttagggataatgaaggagaagaagcagagggcttgcgacacttaaagtatgagtcggggaggctgttgcgtaatgaccacacttccaagcgggtgctgggcgagcgagactttgaaaaggacccccagaatggcattagcgcggccgaggcacacctaaagtctgcagacttagtcacggcctatgagcatactgtgaaggcagaggttaattttcagaccacctttaacaacaatgtgcgtaccctcatcgccagggaagaggtggtcattggcctgatgcacttgtgggactttgtggaggcctttctagaaaacccagtgagcaaggccctcactgcccagctctttcttatagtgcaacactgcagggacgagggggtgctgcgggaaagcttgctaaacattgccgagcctgagagccggtggctggtggacctgttaaacctgttacagactattgtggtgcaagagcgaggcttggcagttggtgaaaaggtagcggctataaactactctgtgattactctaagtaaacactatgcaagaaagatattcaactctgtgtttgtgcccatcgacaaggaagccaagattaacacattttacatgcgcactgttgttaagctgctggtgctgagtgatgaccttggcatgtaccgcaatgagcggattgagagggcagtgtctggtgccaggcagcgggaattaaatgacagggaacttatgcaccgactgcggcaagccttggcttctaacggactgccggagctagagggcgaagacagtgctaatattaggtccaaagaggagtggggggtcggagcgggcggaggcgttgcgagcgcgcgctacccccacctgctagactatgaggaagaggagaaccctgatggctccgtctcatttcaacagcatgagcgcggcgctcaaccccatgaaaatggcggccatgcagagtcagcctacagccgacgacagttgggccgcttctattagtcgcattatgtccctaacggcgggggatcggcacaagttttcatcccagccttttgctaacaggctggatgctatattagaggccgtggtgccttcccgcaaagaccctacccatgaaaaagtgctaactatagtaaatgccctaatagaaaatggtgccattaggcgggacgagggggcgggggtgtatgacgccttgttgcaccgcgtgtccaagtacaacagcttgaatgcccaaagcaacctagagaggttggctggagacgtgagggaggcggtagcgcagcaagtgcgcatcgccacgggcaaccttgggtccctaaccgcacttaatggctttttggcacgcctgcccgccaatgtggagcggggccaggagaactacactggctttgtgtcagcccttaagctgctggtaagcgaggtgcccagtacagaggtgtaccagtctgggcctcactactttttgcaaagcagtcgcaacggcacccagacagttaaccttaccaatgcctttgagaacctaaagcccctttggggagttaaggcccccactatggaaaggctcagcatctcagccctgctcacccccaacactagactgctgcttttgctagtgtctccctttacagacagtgttagcattagcagggactcctacttgggttacctgcttaccctgtacagggaagccttgggccgcaatcatttggatgagcgtactcttgaagaggtcacagaggtcagcagggccatgggcagtgagaacataaacaacttgcaagccacccttaactttttgctaaccaatagacaaaaaagaattcccaaagactactccctaaccccagaggaggagaggatagtgaggtttgtacagcaggccgtgagcttgcgcatgatgcaggaaaacttgagccccactgaagctttggacgtaaccgcggccaacatggagcccagcttctatgccagcaacagggattttattaataagcttatggactactttcaccgcgctgcggctatggctccagactactttttgggggcagtgatgaatccccgctggctcccccctgagggcttctttactggggtttttgactttcctgaaagggacagctacacctgggatggactggatagctctttggagctgacgcgccaggatgccatgcggtttctggaagacaagtttatggacgatgaccagagaacagagtctcgcagcctcagcagagttaccaccccagctagctctaggcggtcctcggtgaccatgccacctgggggctttatgggcatgataaataacaacaaaaacgataacatcctcagggagatagatggattagcagacaagctggctcggtggaaaacatacaagcgtgagtcagaggaggctcgtgtgtccctgcctgcggtagtaaggcctaagcagtacaggccccgctcccctatctccagcgatgacagtgatgatgggatgagcaggccagacccgtttctcaagtttgaggggagtggaaacccctttgcccacctacgccccaagctggggcgtggcctgtaaagtgaataaaatactcaccagagacatgacgctagcgtttttttatagcaatggagttttcgtcgtctcctcccccgtcttatgaaacagtgatggcacaagtgccttcgatcctggcgccgctggtacccccgcggtacaaaggggctacagaaggaagaaacagtatccgttattcccagctgccgcctctgtttgacactacaaagctgtacctcattgacaacaagtcttcagacattcaggccctcaattaccaaaatgaccacagcaactttttaaccactgtggtgcaaaatgctaattacacgcccatggaggctagcacccagtccatccagctggacgagcggtcgcgctggggtggggaatttaagtccattttacacatgaacatgcctaatgtgacagagtacatgtttagcaatagctttaaggcataccttcccgccacagcagacaactttggcaaggtgctcacctatgagtggtacaccctaaccattcccgagggtaactattctgaggttatgcttttggacctgctgaacaacgcggtggtggagaactatctggcccacgggcgccagcataatgttagggaggaggacatgggcctcaagtttgacaccagaaactttcacctgggctttgatcccgagaccaagctggtcatgccgggcttctacactaacgaggccttccaccctgacatagtgctaagtccaggctgtgcggtggactttacccacagcaggctcaacaactttttaggcattagaaaaaggctaccataccaggagggatttgtgattacctgggaggacctgcagggtggcaacattccagccctgttagacctggaaaattacaacccagacattcccggcgccgatatcacaccactgatgtatgactccaagggaaggccttaccatgttggtgaggatcccagcgcgggcagtaccttcacctggtaccgcagctggtttgtggcctataactacggtcctgccgatggcatcaagagcaaaactgtactggtggcccctgacattacttgcggagtggagcaaatttactggagcctgccagatatggcggtggacccagtaacttttacctctagccacaatcccagcagctacccagtggtgggcacggagctgctaccattactgccaagaagcttttacaatggatcttcagtgtacagccagctgttgcaagaaagcaccagtcaaactcaagtgtttaaccgctttccggagaatgcaattcttaaaaggccccctgctcccaccattatcagcatcagcgagaacgtgcccgccctcagtaaccatggcacctgcccctaaaaaacaacattcccggggtgcagcgggtgaccattacggatgcaagaagaagagtgtgtccctacgtgtacaaaagcttgggggtcgttgtgcctcgcgtgctgtccagtaaaaccttctagtgacaaactttttctttttattagcaaagccatggccatcttaatatcaccgactaacaacaccggctggggcctaggcactcacaagctctttggaggagccaaacaaaaatcagatcagcatcccgtttatgtccaagctcactaccgggcctcgtggggcagcaaagggcgccgccgccgtcaaggccgagcccgcggggcccctcttgaccctaaaactgaagcagaaatggttgccaccatcgacgaggtggcgcgcaacggtcccccggcggcgcgcttggtgctggaggctgcccggagagtgggggcttacaacctccgtcgagcgcgcaagctaacccctgcggggcgtgccatgatggccatgcgcgcgcgtcagatggtaaagcaggctaagaaaaggaagcgcagggttcgctttagacaataaagtctgtcctcacatacacacatacccgagtgatggcctatttttttactgaagcctcatcgccatggcagcaatcagtcgcgccatcaagcaggaactgctggaagacctcaaacctgaaatctatctgccacccaagtccaccaaacggagaactaaagttaaaacagaggaaaaagttgatgtgaaaaccctagtcaatgccaagagcagaaagcgcagggctgccaaggatgagctggaagaggatgtagagtttgttaggcgctttgctccccggcgtccctaccagtggcggggtcggcgcgtgcgcgctctgcctaggcccggggtgcccgtggtgtttactcccggccagaggtcgggcaccgcctcaaaaaggtcttatgacgaagtgtacgcagatgaggatgtactggaccaggcgggaaacatgattaatgagtttgcttatggcaagcgagtcagggtgcttacccacaaaaaccccactccctcacaagtgcccatcaccccccaggagcccgtggcgcgtcctggcgaggcacacctgctacccacagtgcaagtgctggctccgcgaggctctaggcgggaaaccatgttgcccgtaaccaagtccgaggggggagacgtgaaagtggaaaacaagggctttgagcaaatcacccccgggctgggggtgcagactgtagacatcaaagtgcccgtgaagcgcaaaggcgatgcagaggatgaaataataaagagggttaagatggagcttgaaccatatgagaccactatgaaaatggagtactctgaggagccccaggttgaggcttttgacacagggatagagcccagctctttttttgaggtgcgctctcaagcgcgtcccatcgctgtggctaggaagcgccgcaccgcagctgccagcgcccccgctgtagaggtgatggaggttcagcaaagcaaccctgccaccccagctaccgctgccgccagaaccgcgaccgcgctgggcccgcgtttggctcgtcggccttcaagatgggggcccgccaatgccatctttccagattacaaataccaccccagcatcacggctaggaaaatgaggggccccaagcctacaggcaagatcagccgctgggggcctgccaacagcatcctgcctgaggtgcggctgcaccccagcatggtgtctgcggtgaccagagctgcccctcgcagagtcaccaaaacccggagacgccggcgtgccagaacccgccaggcttttgtgctgcccgcgcgcaccaagacgggcgcgctgctttcccagaacgtgcgctaccacccttccatctccttgctccgccgagcctaatctgtatgttttgctttgcgcaaggatggccggcaggaatgtcacccttcgtctgagagtcccagtgcgcaccaaaattacaggcgcaggtcgtcggcgcggacggcgcccacgcggcatacgctgcgggcgcatgagaggaggctttttgccagccctcatccctttgatagctgccgccattggggcagtaccaggaatagcttcggtagcgctccaagctgcccggcactagctttttacagattgactcactcccatttttcttgtgccagaagaagacaaaatggatgctgtcaatttttccatcttggcaccacgctacggggcccaccctatgatgagtggttggtctggcatcggcaccagcggcatgaacggcggggcctttaactgggggggcatttggagcggcatcaaaaactttggcagtaatgtaaaaagctggggcagcaaggcttggaacagccaaacgggcaagctgctcaggcaaaagcttaatgacaccaaagttagagaaaagctggtggagggcatttccaccggggtgcacggggcacttgatatcgccaaccaggagttagcaaagcaaatagagcggcgtttggagcgtcagcagcccctagagccggaggtggaggtagaggaggaagtagtggacataaaacccgaagcgcaggccccgctggtggtacaaatacccaaaaagcgcccccgcgacgaagacttgctcataactgcggacgagcctccctcctatgaagagtccatcaaaaccatggcacccctgatgcccatgacccgcccgcacccttccatggccaaacctgttttggtagaccgtcccaccaccttggagctaaagccctctgaccagccgccagcctactcccccccggcgcccagcgccgtcagggtcacagtgccctctaacattcccgtggtcactcctgccagaagcagaggctggcaaggaactctggccaacattgtaggagtgggtctaagtaacgtgaagcgccgtagatgcttctaactgtgattaaatatactcaagtgacttgaagtttgccgaccctgtctttttttgaccgcgccagctgagaagatggcaaccccgtcgatgctgccacaatggtcttacatgcacattgctggccaggacgccgccgaatacttgtctcccgccctggttcagtttgcccaagcaaccagttcttactttaagttggacaacaagttcagaaaccccactgtggcccccacccacgatgtgaccactgagaggtcgcagcgcttgcagctgcgctttgtgccagtcatgcaagaggatggccagtacacttacaaaacccgcttccagcttgcggtgggagacaacagggtgctggacatggccagtacttacttcgatatcaggggtaccctagacagaggcccctccttcaagccttacagcggcaccgcctacaatgccctcgcccccaaggccggggctaacaactgtctttttaatggacagggtgccaatattaacactttagcccaggtgccctctgcaggtgccataactgtgaatggccaagctgctgtcacaaacaatacctaccagccagagccccagctgggccctgaaagctgggtcgatggcagcctagcagagctgggggatgcgtctggccgtgcccttaaggcttcaaccccgcgcatgccttgctatggttcctatgctccccccaccaacgaaaatggaggtcaagcaactggtccagtggaatccagattttataaggtgaccaccaacaataacaatgaagcagatgccatgctatacactgaagatgtaaacctgcaggccccagacacccacctggtgcaccaagtgccagagggtcaggttacaggggtgcaagggctgggccagcaggctgcgcccaacaggccgaactacataggcttcagggacaacttcataggcctcatgtactacaatagtaatggaaacctaggggtgctggcgggtcagtcatctcagctcaatgccgtggtggacttgcaagacagaaacacagagctctcttaccagctgctgctggatgccctcacagacaggtcccgctacttttccatgtggaaccaggctgtagatagctatgaccaggatgttaggattattgacaaccatggcgtggaagatgatatgcccaactattgctacccactgagcggcatggggcccctaacaaacatgaccaccatgaaggttaacaaccaaaactttcaggcagaaaataccaatgtggggcccattcaaaagattggttttggaaatgttgaggccatggaaatcaacctcaatgccaacctcttcaaaagcttcctttactccaatgtggccttatacttgcctgatgcctttaaatacacacctgaaaacattgtggcccctgccaatgtgaatacctatgcttacatgaatgttagattacccgccgccaaccttatagatacctttgtaaatattggcgccagatggtcaccagatgtaatggacactgttaatcctttcaaccaccacagaaatgcaggactccgctaccgttcacaactgcttggcaatggccgctattgctcgttccatattcaggtccctcaaaaattttttgcaatcaaaaatctcctcctactgcctggaacgtacacgtacgagtggtctttcagaaaggatgtaaacatgatccttcaaagcagcttgggcaatgacctccgagtggatggggccaccatcaacattcagagcatcaacctatatgcaagctttttcccaatggcacacaacactgcctccactctggaagccatgctgcgcaacgatgtaaatgaccagtcctttgcagactacctgtcttctgccaacatgctttatcccatccctgccaacactactaacctgccaatctccattcccgccagaaactgggcgggatttagagggtggagctttaccagaattaagcaacgagaaactcctgccctgggctcgccttatgatccctacttcacttattcgggcagtattccatatctggatgcaactttttacctcagccacacctttagaagagtttccatcatgtttgactcttccgtgtcttggcctggcaatgacaggctgcttacccccaatgagtttgagattaaaaggtatgtagacggtgaaggttacaatgtggcccagtccaacatgacaaaagactggttcatggttcaaatgctagcccactacaacatcggctaccaaggctaccacctgccagaaagctacaaggacagaatgtactccttcctaagaaactttgagcccatgtgcagacagttggtggacgtggccaactatgctgcctaccagccggttaccgtgggccaccagcataacaattctggttatgctagcgccctttcggcctttaacccgcgtgaggggcacccatacccagcaaactggccttacccactcattggagccaatgcagtacccactgtcacccagaaaaagttcctctgcgacaggtccctgtggcgcatcccattctcctccaactttatgtctatgggaaccctcactgacctgggccaaaacctgctgtactctaactccgcccacgcccttgacatgacttttgaggttgatgccatgaatgagcccactctgttgtacgttttgtttgaagtgttcgacgtggcacgcgtccatcagccccaccggggggtgattgaggtagtgtacctcagaactcccttctccgccggcaacgccaccacctaagctaatatggcggaaggagggtcctcagaagaagagctgcgagccatagtacataacttaggcgtgtctccatttttcttgggcacctttgacaagaggtttccgggtttcatctcttcacaaagaatggcctgcgccatagtaaacaccgcagccgagaaacggggggcgtgcactggctggccatggcctggaacccccgctcaaaaacgttttacatgtttgacccctttggattttcagacagtaagctcaagcaagtgtacagctttgagtatgagggcctactcaggcgcagcgccatagcctccagcccggataggtgtgtcactttggccaaaagcaatgaaaccattcagggtcccaatagcgccgcctgtggactgttttgttgtatgtttttgcatgcctttgtcaactggcctgacgacccctttgaccacaatcccaccatgggacccctcaagagcgtgcctaactacaaactcaatgatcccacagtgcagtatgtgctctggggaaatcaagaaaagctgtataagtttttggaaaaacactctgcttactttcgcgcccacgccgctgcaattaaagccaggactgcttttaataaattgaaacaataaaccgtttattcgaaaaatataaatggtttacagtgtgattattaaaaaaggacaaggtcatcgtcatgctggccctggggcaaaatggtattttggtacctgtgttcatcggaccagtggaactctgggaatttaattgggggcctctcgcccactgtggacgtccatatttgcttagcaatttggacacatgacaccaggtccacgctggaaatcttaaaatcacaatttttttggggggcggccttgctgttgcggtacactggattgcaacattgaaacactagcatggcggggttattaagggtggctagcattttttggtcatccacttcactcttgtcaatgtgactagcgcttgcaagagagaatggggtaatcttgcaagtttgcctgcccagcaaaggaaggttactgttccaattgcactcacacttgagtggcattagcaagtgactctcagcatttggcatagaggggtagcaagccttaagaaaagccatgatctgctgaaaggccataatagctttgggtccatcggtataaaacataccgcaagaggccccgctaaagttgcccccgctcatattaacatccatgctgcagcagaaagcatcctcattcttaaactgtactacattccttccccaccggttagaaacaattttggttttttctgggttttccttaagggcccgctgggcattttcactgttaacatccatttctactagctgctctttttggatcatgggcgacccgtgcagacacataaggctgctctggcatccatgttgccacaccacacacccgctggggttaaccccaggggcaagctctgctgctttgatcacaaaatcaagcagcaggcgccctgccacgtgcaaaaaagttttctggttggtaaaggtgtaagtaacgtgcgttttagaggtggtaatgtaagcctggactgcttttttaaagcactccagcgtgccctcgtctggaagcaaggttagcttgctgtggtctacctggtacgcggtgagaaccttaagggcaagctccatggcagactgccacttgtgctcactagctttaaaaaacttagaggccactagccccgagggcgcgttgggcttgctccaaacggacttttgcttgggtgggataaataccacatttccctccttgtcagtgtccaccgtttggccacaaaaggtctgcactacaatgctcccgggatccccccgcgccttgagggtggcggtggcttgacgggctatttcgaccatggcttggtggccccttggctcctcaacggctttccgctggcgcttctttgggggcgccgtaggcggtgccacaggaacctcttcatcgctggagctttcagagattgcaacaaccttcttgtggctcatctttttcctagatgtcagaagagcccgtcagtgggactaccgtggagacaaaggaggacattcacacacctccaaactcccctgtcctcgagacaatctctctaagccccgaacccgaggctgaggcctgcccaaatacagacaaatacctaagcaccaatttgctttgcaaacacctgcaacgtcaaagcgcaattgttctggacagtatcaaagatcatctacaagtgcccaccagtgtatcagagctaagctgcgcctacgaacgaagcttattctccccaaacaccccaccaaggcaacaaagcaatggaacgtgtgaagcaaatcctaaacttaacttttaccctacttttttggtgcctgaaactttggcaacttatcacatcttttttgttaatcaaaagataccagtatcttgcagagctaacagagctaaagcagacagagctcttgctttgcaagagggagattgcttacctgactatgaaacaatggacacagtcagccgggtgtttgagggtttaggcggtgaggtggttgcggatcacgcgctgcaaaataatgactctgtattagtggagcttaaggaagacaacccccgtttggctgtggtcaaaagaaacctcagcgtctcccactttgcctaccccgccgtgcacctgccgccaaaggtcataaccactgtcatggacaacctgctagctaaaagggccgcccccagcgccgacgtctcagagctagatcccgaggggggccaagaggtggtgtctgacaccgagctaagtaagtggctaaacacctctgaccccgaggccttagaaaagcagcgcaagacagtaatgggaagcgtgctggttactgtagtgcttgagtgcatgcaaaggcttttcacctcagaagaaatggtgaaaaaaattggggaggccctccactacacctttaggcacggctacgtatctctggcttgcaagatttctaatgtagagctgaccaacgtggtcacctatatgggcatcctgcatgaaaaccgcctgggtcaaaccaccctgcatcacacagtgcagggcgaggcccgtcgagactacattagggactccatcttcctcatccttatccatgcctggcaaaccgcgatgggcatctggcagcagtgcctcgaagaggaaaaccttaaagaactagccaaactgctgcaaaggattaaaaagccattgtacactgaaacctcccagcgcctcatgggcaagcgcctggcaaacgtagtcttcccacccaaactgctagaaaccttccacaagggccttcctgatattgttagccaaagcatgatgcaaaactttaggtctttcattttggagaggtccggcatcctgccctccatgacctgcgcgctgcccacagattttgtaccaattcactttaaagagtgcccccccactatgtggccctacacctacttgctcaaattagcaaacttctttatgtatcacaatgacctttgctatgatgtgggaggcgagggcctgctagagcactactgccgctgcaacctctgcaccccccaccgttgcctagccaccaaccccgccatgcttaatgaaactcagctaataggtacctttgatattcggggtcccggcggagaaaacggagcagagtcttcctctggccttaagctcaccgctggaatgtggacttccgcgtttctgcgaaaatttgaaagctctgattatcacgcccacaaaattcagttttatgaaaaccaatcaaagcccccctcagtggagcccactccctgcgtcatcacccaaagtagcattttggcccaattgcatgacattaaaaaagcaagggaggagtttctgctcaaaagcgggcagggtcagtatctagaccctcagactggggagccgctcaacgctgcggatccttccgtagaaagcagccatgagttccaaggagatggaagacacagaaaccctaagcgtggaagacatttccggcagcgaggaggacctagaaagcctcccagagtccatgccggagcagagcgagatgtcagaggaacgagctcctagatgggaccaaaaagagaagcctctgggtaagcagccccgcaactaccactcctggagagcgcataagtttaagattcttagctgcctgggcgccagtgggaacaatgtagcctttaccagaagatacatgctctttcgcgagggagttaaccttcccaacaacatcattcactactataactctcgctaccgcagcaaaacagaagctcaagcgcagaccgaccccactaaagggcccaaagccagccgccggtgaggacaaccaccagacaaggcagctgcgcaaccgcatttttccaacactgtacgccatcttccaacaaagcagaggctcccccacggcattaaaaataaaaaacagatccctcagatccctcctaagaagctgtctctaccacaagtcagaggagcagctgctgcgcacgcgaaacgacgccgaagctttgctcaacaaatactgtcaaggactcgagtccggcacagactgagcaatgtctaaagaaataccaaccccttatatgtggagctaccaaccgcaaacgggacacgccgccggcgcctcccaggactactccacccaaatgaattggtttagtgctgggccatcaatgattagtcaagtttatggcattagagacttgcgcaacaaagttttgataacccaggcagaaataaccaaaactcccagaacaataatggatccgccaatttggccagctgccatgcttgttcaggaagccgccccacccaaaacggtcactctgcccagaaaccacaccctagaacaggctatgaccaactctggggcgcagctagcgggaggacgacagctgtgcccctcccaaataggtataaaaagcccagtgctggctggcacgggcattcagcttagcgaagacatccccagcgcctcctggatcaggcccgacggcatattccagctaggaggggggtctcgctcgtccttcagcccaacgcaagcattcctcaccctgcaacaggcatcctcgacgccgcgcgcaggaggcgtgggcacctaccagtttgtgcgcgaatttgtgccagaggtataccttaaccctttttcaggaccaccggacacctttcctgatcagttcattcctaactacgacattgtaaccaactctgtcgatggctatgactgaggagagcatggaccaggtttaaacgcgatcgcctcgaggcgcgccggccggcctcagccttctaatgggactaaacaacaaaatcaggcccatgtagcttgtcaaataaacttacctaatttttgctaagacgtctgggtcctgcgtttctatgtccaccaaagtcccctcttcccagctttggtacttccacttgtgcgcgcgagccagcttgcggatgtgcttgaaagataatgtggtctctcccaacagcttcccgttcaccagcaccagggccatgaagcggacacgaagagctctacctgcaaattatgaccctgtatatccatacgacgcccccgggtcttccacacaaccccctttttttaataacaagcaaggtctcactgagtcacccccaggaaccctggctgtcaatgtttcccctccactaaccttttctacgttaggtgccattaaactttccacaggtcccggactcaccctcaacgagggcaagttacaagccagcttagggcccggcctcatcacaaataccgagggccaaatcactgttgaaaatgtcaacaaggttttgtcttttacctccccattacataaaaatgaaaacactgtatccctagcgctaggagatgggttagaagatgaaaatggcacccttaaagtgaccttccctactccccctcccccgctacaattctcccctcccctcacaaaaacaggtggtactgtttccttgcccctgcaagactccatgcaagtgacaaatggaaaactgggcgttaagcctaccacctacgcacctcccttgaaaaaaactgaccagcaagttagcctccaagtaggctcgggtctcaccgtgattaacgaacagttgcaagctgtccagcctcccgcaaccacctacaacgagcctctttccaaaactgacaattctgtttctctgcaagtaggtgccggccttgccgtgcagagcggagctttggtggcaacccctcccccgcctctcacctttacatcacccctagaaaaaaatgaaaacacagtgtcgctacaagtaggcgcgggcttgtctgtacaaaacaacgccctagtagccacacctcccccacccttaacctttgcctatcccttagtaaaaaatgacaaccatgtagctctaagtgctggaagtggtttaagaatatctggaggcagcctcacggtggccactggacctggcctttcccatcaaaatggaacaataggggctgtagtaggtgcaggcctcaagtttgaaaacaatgccattcttgcaaaactaggcaacggtctaaccattagagatggcgctattgaagcaacccaacccccagctgcccccataactctgtggactgggcctggccctagcattaatggctttattaaggacactccagtaattaggtgctttatatgcctaaccagagacagcaacttagtcacagtaaatgctagctttgtgggagagggggggtatcgaatagtcagccctacccagtcacaatttagcctaattatggagtttgatcagtttggacagcttatgtccacaggaaacattaactccaccactacttggggagaaaagccctggggcaataacactgtacagccacgcccaagccacacctggaaactgtgcatgcctaacagagaagtttactccactcccgccgccaccatctcccgctgtggactagacagcattgcagtcgacggtgcccccagcagaagtatcgactgcatgctaattattaacaaaccaaaaggcgttgccacttacacccttacctttaggtttttaaactttaacagactaagcggaggtaccctgtttaaaactgatgtcttaacctttacctatgtaggcgaaaatcaataaaaccagaaaaaaataagtttaaaagctttatttttcatacacgcgagcggtaaggctgccgccttcaggaaaagttactctgtaaacagttctttcacaacagcacaaaacataggtattagttaacagttcatttgggctataataatatacattttcttgggtggcaaagcaagggtcggtaatctcaacaaaaccatcaactggaatgcaagaatagtccagcacggtgggttcaatctaaaaatgaagaaacgcgttgaggttcactaagcacaggttttgaatctgtcggcagcgtccatgcatcatagcttgtctcaaagcagattgtcttctttcctctgccttggaagtggtttggtgaagcactacaggtgtcttttcaacctctttcagcacccgctctattacagatctcacccacacagcacagtttttaagagaacaatagttttgaaggctacaagatttacacttaagcaccagccagtaattataagtgcttttaagaactacccctagctcagggttaatgcaccttttaatggcctccatgcaggctttatggacagttctaaaaaaagacagtctaaaataaatgtagtgagtgtttctaaatataatactccccacatagttaatttcatcaggcctgctagaatttacaaactctcggtaccacatatactttttattcatagccccacccttaataaagtcctcaatcactttctgaaccacatgcttgctagccatgcattgtaaagacaagctgttagagcagtgacagtgtactcgccacgtttgagcctctgccaggcagcagtgcttagttactatcaactcaatacccgcattgcatgtaaaccccccaaagagcagtttttcatgcctgtgtagcacatcatcccacaaaataggaatttcatagcataaagcaaagcaattacaatatttaggaactctcaccacagcagtcacgtgacatgttgtctcagcagtgcagttgccttccatcctacaattatgaacaaaaactaaacacttctaacaaagatacagtgacaatctcccttcctctaaaagcattgtttacattagggtgattattaacaacgtcagaaatttctttaattaaagtgcctttaaaatgtgcaagagcatcatcatactcaaaaccaagctgagagtaaaagaccaccttaaaagtaatcccaggcttgtttttatcaacagccttaaacatgctttcacaaaatatagaagcagtaacatcatcaatggtgtcgaagagaaactccataggagactccagcattgatccaagctctctaacaaaatcttcctcaaaatgaataatgccctttacacaaacgcggggcagacgatggtgggccatcgcgtcaacctgaaacacattttacagtaaacaaagctagctccgcagtggtaaagtcatgcccatgggtgaggccaaaatccttaaaaaagctacctaagtagttggtcatcccctcagttaaaaagttttgcagctgggtggtgcataccacatagtgccagcttatagctacaaagacctccatcccctccttagcagacagctcttgcacacacgcagtaactatccaccgcttaagaaaagctttaagcccagcgcacataacagctccaatgtttttatccaaggagagcaaaatttcagcaagcgcaggctcaacagtaatagtgaagcagaggcatttcagacgaggctcactagctgcagtcgccatttatgaggtctgcaataaaaaacaactcatcagcagctgaaaaagtgcactttgacctcattaagccactgcatatgcaagtcctcatctatgccgcagcccagaccctcaatccagccccgaatgtacactttaataagagattcaacctcttcttttagcaaagtacacatgctgtttggactagtatacacaatagaagtcacaatgaggggcccgctgtggctggaaagcctgcgcacagcccgaaggttaaaaatggactgtaacagcattgaaaccccgcgacacaggtcagtctcgcggtcttgatctcttattatagcgaccaaatggtccttcagagtgatgttgcactcatagaagtaggcagctccggcagccattctgcaaaataacaaaacaccactaagcatagcaccatcaccaagcatgaaaacaggtaaaaacaaaagcaacacttacttattcagcagtcacaagaatgttgggctcccaagtgacagacaagcctaatgcaaggtgggcacagtcctccggaataagttgacaaaagtcacgccgcaaagcttcctgaagagaaacggcggtagcctggatatctgcaacggacccaaaaccttcagtgtcacttccaataaacagataaaactctaaatagtccccacttaaaaccgaaacagccgcggcaaaggtaggacacggacgcacttcctgagccctaataaggctaaacaccacacggcgcagttcagaaggcaaaaagtctgtaagctctagctgagcacacacactctccactagactcttgtgaagcctcagacaaaaacatgctcccatagacactcctaaagctgccattgtactcacggacggctggctgtcagaggagagctatgaggatgaaatgccaagcacagcgtttatatagtcctcaaagtagggcgtgtggaaaacgaaaaggaatataacggggcgtttgaggaagtggtgccaagtacagtcataaaatgtgggcgcgtggtaaatgttaagtgcagtttccctttggcggttggcccggaaagttcacaaaaagtacagcacgtccttgtcaccgtgtcaaccacaaaaccacaaataggcacaacgcccaaaaacccggggcgccggccaaaagtccgcggaactcgccctgtcgtaaaaccacgcctttgacgtcactggacattcccgtgggaacaccctgaccagggcgtgacctgaacctgaccgtcccatgaccccgccccttgcaacgcccaaatttaagccacacctctttgtcctgtatattattgatgatg

SEQ ID NO: 4 - DNA, 617 bp, synthetic construct
  source 1..617: mol_type = other DNA
acattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagagaacccactgcttactggc

SEQ ID NO: 5 - DNA, 903 bp, synthetic construct
  source 1..903: mol_type = other DNA
aatctaacatggctgctatagcttactgactagaagttaagtgcacacttcctaaaagaaggctttgacacaagccacttcagttccctcctcattttcttgtccccattcctctctctgtagaattctgagatttcaattcagttttatacagaaaccacattactgtaagccctacaaagttatggcaatatagctatatggagtcaagtaatgtaggttatttttttcccaatggtgctggtgaaggtggcaattatgtagctatacttagcagactgaggaaattctgctagagtcagcatttgtctcttcattgctatgaaacagtaatggaaaaataaacaaaaacaaaaggcaaacactatgcataattccctcagatcatattaacatgtgatgttggagtaaattgttataaccccattttggaaatacttaccttaattaactatgatttccttaaaataatgcagtatttacaatctatatgaaagcactatatgggacacatggtatgatggaacagtgcacccaagagacaccaagaacattcctgtctgtggcagtcttttctctatacagaggcatttagtctcaattgctcagagttattttaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 6 - DNA, 891 bp, synthetic construct
  source 1..891: mol_type = other DNA
ttgtcactttgttactctacagtgttgcctggagttcgatacttcattactctatagtggggtgaagaagttcaccttcttattttcatttccttccctcaatgatttcttcagagctagctcttaccagctagaaattcttcaaacgacactcgtgccttccttcacacaggttgaactatttgtctctaatgccctaaagtactggtgttcaatcttccaggcacttccaatgatctgaaatctgacctgcttaggtcagctggctctgagattatggtattctagtcctcaaaccaacctgttggctcgttggttttgtaccaaacacactgacttacatagctcaaaataccactggccttttaaaaatggcatatcacattccaggggaggatcaaaactgctggctggtgatatttgtcaagtctctcaaagttgcactttccaggattttcaattcactgaattcttagacagacatgtttatgtgaaagaattctttatatattttttctcctctttgagtgggcaaatgaaaatcttgacctctgggttccttattttatttgactctctgtagtatttaaatcttaaaattttcctaaacttcctgcagcccgggctgggcataaaagtcagggcagagccatctattgcttacatttgcttctagcctgcaagtcgaggagcgcagccttccagaagcagagcgcggcgccttaagctgcagaagttggtcgtgaggcactgggcacgtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 7 - DNA, 1179 bp, synthetic construct
  source 1..1179: mol_type = other DNA
ggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 8 - DNA, 448 bp, synthetic construct
  source 1..448: mol_type = other DNA
agtgcaagtgggttttaggaccaggatgaggcggggtgggggtgcctacctgacgaccgaccccgacccactggacaagcacccaacccccattccccaaattgcgcatcccctatcagagagggggaggggaaacaggatgcggcgaggcgcgtgcgcactgccagcttcagcaccgcggacagtgccttcgcccccgcctggcggcgcgcgccaccgccgcctcagcactgaaggcgcgctgacgtcactcgccggtcccccgcaaactccccttcccggccaccttggtcgcgtccgcgccgccgccggcccagccggaccgcaccacgcgaggcgcgagataggggggcacgggcgcgaccatctgcgctgcggcgccggcgactcagcgctgcctcagtctgcggtgggcagcggaggagtcgtgtcgtgcctgagagcgcag

SEQ ID NO: 9 - DNA, 823 bp, synthetic construct
  source 1..823: mol_type = other DNA
tatacactcacagtggtttggcatatatttggtgaaattttttaaggaaaaattagtgttggtttcgatatatggtagctttttctctaacataatttgaataattcagcaaagccctactaccagctgtacttctgcagcctcttccattctttccagcattataattttggttaattttcaattttaggtcctacgtctctgcaatttgtgtatgaataacagaataatttccctcttttgtttcgcctttcctgttcctgaatctaaataaagatggctttttagtattaaaagtggaagaaaattacaggtaattatctttgacggtaaaaacgctgtaatcagcgggctacatgaaaaattactctaattatggctgcatttaagagaatggaaaaaaaccttcttgtggataaaaaccttaaattgtccccaatgtctgcttcaaattggatggcactgcagctggaggctttgttcagaattgatcctggggagctacgaacccaaagtttcacagtaggaagcttgatatcgaattcctgcagcccgggctgggcataaaagtcagggcagagccatctattgcttacatttgcttctagcctgcaggtcgaggagcgcagccttccagaagcagagcgcggcgccttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 10 - DNA, 1194 bp, synthetic construct
  source 1..1194: mol_type = other DNA
ttaaggggacagtaaaggtgatggcaggaaggcagcccccggaggtcaaaggctgggcacgcgggaggagaggccagagtcagaggctgcgggtatctcagatatgaaggaaagatgagagaggctcaggaagaggtaagaaaagacacaagagaccagagaagggagaagaattagagagggaggcagaggaccgctgtctctacagacatagctggtagagactgggaggaagggatgaaccctgagcgcatgaagggaaggaggtggctggtggtatatggaggatgtagctgggccagggaaaagatcctgcactaaaaatctgaagctaaaaataacaggacacggggtggagaggcgaaaggagggcagattgaggcagagagactgagaggcctggggatgtgggcattccggtagggcacacagttcacttgtcttctctttttccaggaggccaaagatgctgacctcaagaactcataataccccagtggggaccaccgcattcatagccctgttacaagaagtgggagatgttcctttttgtcccagactggaaatccattacatcccgaggctcaggttctgtggtggtcatctctgtgtggcttgttctgtgggcctacctaaagtcctaagcacagctctcaagcagatccgaggcgactaagatgctagtaggggttgtctggagagaagagccgaggaggtgggctgtgatggatcagttcagctttcaaataaaaaggcgtttttatattctgtgtcgagttcgtgaacccctgtggtgggcttctccatctgtctgggttagtacctgccactatactggaataaggggacgcctgcttccctcgagttggctggacaaggttatgagcatccgtgtacttatggggttgccagcttggtcctggatcgcccgggcccttcccccacccgttcggttccccaccaccacccgcgctcgtacgtgcgtctccgcctgcagctcttgactcatcggggcccccgggtcacatgcgctcgctcggctctataggcgccgccccctgcccaccccccgcccgcgctgggagccgcagccgccgccactcctgctctctctgcgccgccgccgtcaccaccgccaccgccaccggctgagtctgcagtcctccgaaacgggccctctagactcgagcggcc

SEQ ID NO: 11 - protein, 2009 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEVIIDKPATDDNGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 12 - DNA, 6030 bp, Homo sapiens
  source 1..6030: mol_type = genomic DNA
atggagcaaacagtgcttgtaccaccaggacctgacagcttcaacttcttcaccagagaatctcttgcggctattgaaagacgcattgcagaagaaaaggcaaagaatcccaaaccagacaaaaaagatgacgacgaaaatggcccaaagccaaatagtgacttggaagctggaaagaaccttccatttatttatggagacattcctccagagatggtgtcagagcccctggaggacctggacccctactatatcaataagaaaacttttatagtattgaataaagggaaggccatcttccggttcagtgccacctctgccctgtacattttaactcccttcaatcctcttaggaaaatagctattaagattttggtacattcattattcagcatgctaattatgtgcactattttgacaaactgtgtgtttatgacaatgagtaaccctcctgattggacaaagaatgtagaatacaccttcacaggaatatatacttttgaatcacttataaaaattattgcaaggggattctgtttagaagattttactttccttcgggatccatggaactggctcgatttcactgtcattacatttgcgtacgtcacagagtttgtggacctgggcaatgtctcggcattgagaacattcagagttctccgagcattgaagacgatttcagtcattccaggcctgaaaaccattgtgggagccctgatccagtctgtgaagaagctctcagatgtaatgatcctgactgtgttctgtctgagcgtatttgctctaattgggctgcagctgttcatgggcaacctgaggaataaatgtatacaatggcctcccaccaatgcttccttggaggaacatagtatagaaaagaatataactgtgaattataatggtacacttataaatgaaactgtctttgagtttgactggaagtcatatattcaagattcaagatatcattatttcctggagggttttttagatgcactactatgtggaaatagctctgatgcaggccaatgtccagagggatatatgtgtgtgaaagctggtagaaatcccaattatggctacacaagctttgataccttcagttgggcttttttgtccttgtttcgactaatgactcaggacttctgggaaaatctttatcaactgacattacgtgctgctgggaaaacgtacatgatattttttgtattggtcattttcttgggctcattctacctaataaatttgatcctggctgtggtggccatggcctacgaggaacagaatcaggccaccttggaagaagcagaacagaaagaggccgaatttcagcagatgattgaacagcttaaaaagcaacaggaggcagctcagcaggcagcaacggcaactgcctcagaacattccagagagcccagtgcagcaggcaggctctcagacagctcatctgaagcctctaagttgagttccaagagtgctaaggaaagaagaaatcggaggaagaaaagaaaacagaaagagcagtctggtggggaagagaaagatgaggatgaattccaaaaatctgaatctgaggacagcatcaggaggaaaggttttcgcttctccattgaagggaaccgattgacatatgaaaagaggtactcctccccacaccagtctttgttgagcatccgtggctccctattttcaccaaggcgaaatagcagaacaagccttttcagctttagagggcgagcaaaggatgtgggatctgagaacgacttcgcagatgatgagcacagcacctttgaggataacgagagccgtagagattccttgtttgtgccccgacgacacggagagagacgcaacagcaacctgagtcagaccagtaggtcatcccggatgctggcagtgtttccagcgaatgggaagatgcacagcactgtggattgcaatggtgtggtttccttggttggtggaccttcagttcctacatcgcctgttggacagcttctgccagaggtgataatagataagccagctactgatgacaatggaacaaccactgaaactgaaatgagaaagagaaggtcaagttctttccacgtttccatggactttctagaagatccttcccaaaggcaacgagcaatgagtatagccagcattctaacaaatacagtagaagaacttgaagaatccaggcagaaatgcccaccctgttggtataaattttccaacatattcttaatctgggactgttctccatattggttaaaagtgaaacatgttgtcaacctggttgtgatggacccatttgttgacctggccatcaccatctgtattgtcttaaatactcttttcatggccatggagcactatccaatgacggaccatttcaataatgtgcttacagtaggaaacttggttttcactgggatctttacagcagaaatgtttctgaaaattattgccatggatccttactattatttccaagaaggctggaatatctttgacggttttattgtgacgcttagcctggtagaacttggactcgccaatgtggaaggattatctgttctccgttcatttcgattgctgcgagttttcaagttggcaaaatcttggccaacgttaaatatgctaataaagatcatcggcaattccgtgggggctctgggaaatttaaccctcgtcttggccatcatcgtcttcatttttgccgtggtcggcatgcagctctttggtaaaagctacaaagattgtgtctgcaagatcgccagtgattgtcaactcccacgctggcacatgaatgacttcttccactccttcctgattgtgttccgcgtgctgtgtggggagtggatagagaccatgtgggactgtatggaggttgctggtcaagccatgtgccttactgtcttcatgatggtcatggtgattggaaacctagtggtcctgaatctctttctggccttgcttctgagctcatttagtgcagacaaccttgcagccactgatgatgataatgaaatgaataatctccaaattgctgtggataggatgcacaaaggagtagcttatgtgaaaagaaaaatatatgaatttattcaacagtccttcattaggaaacaaaagattttagatgaaattaaaccacttgatgatctaaacaacaagaaagacagttgtatgtccaatcatacagcagaaattgggaaagatcttgactatcttaaagatgtaaatggaactacaagtggtataggaactggcagcagtgttgaaaaatacattattgatgaaagtgattacatgtcattcataaacaaccccagtcttactgtgactgtaccaattgctgtaggagaatctgactttgaaaatttaaacacggaagactttagtagtgaatcggatctggaagaaagcaaagagaaactgaatgaaagcagtagctcatcagaaggtagcactgtggacatcggcgcacctgtagaagaacagcccgtagtggaacctgaagaaactcttgaaccagaagcttgtttcactgaaggctgtgtacaaagattcaagtgttgtcaaatcaatgtggaagaaggcagaggaaaacaatggtggaacctgagaaggacgtgtttccgaatagttgaacataactggtttgagaccttcattgttttcatgattctccttagtagtggtgctctggcatttgaagatatatatattgatcagcgaaagacgattaagacgatgttggaatatgctgacaaggttttcacttacattttcattctggaaatgcttctaaaatgggtggcatatggctatcaaacatatttcaccaatgcctggtgttggctggacttcttaattgttgatgtttcattggtcagtttaacagcaaatgccttgggttactcagaacttggagccatcaaatctctcaggacactaagagctctgagacctctaagagccttatctcgatttgaagggatgagggtggttgtgaatgcccttttaggagcaattccatccatcatgaatgtgcttctggtttgtcttatattctggctaattttcagcatcatgggcgtaaatttgtttgctggcaaattctaccactgtattaacaccacaactggtgacaggtttgacatcgaagacgtgaataatcatactgattgcctaaaactaatagaaagaaatgagactgctcgatggaaaaatgtgaaagtaaactttgataatgtaggatttgggtatctctctttgcttcaagttgccacattcaaaggatggatggatataatgtatgcagcagttgattccagaaatgtggaactccagcctaagtatgaagaaagtctgtacatgtatctttactttgttattttcatcatctttgggtccttcttcaccttgaacctgtttattggtgtcatcatagataatttcaaccagcagaaaaagaagtttggaggtcaagacatctttatgacagaagaacagaagaaatactataatgcaatgaaaaaattaggatcgaaaaaaccgcaaaagcctatacctcgaccaggaaacaaatttcaaggaatggtctttgacttcgtaaccagacaagtttttgacataagcatcatgattctcatctgtcttaacatggtcacaatgatggtggaaacagatgaccagagtgaatatgtgactaccattttgtcacgcatcaatctggtgttcattgtgctatttactggagagtgtgtactgaaactcatctctctacgccattattattttaccattggatggaatatttttgattttgtggttgtcattctctccattgtaggtatgtttcttgccgagctgatagaaaagtatttcgtgtcccctaccctgttccgagtgatccgtcttgctaggattggccgaatcctacgtctgatcaaaggagcaaaggggatccgcacgctgctctttgctttgatgatgtcccttcctgcgttgtttaacatcggcctcctactcttcctagtcatgttcatctacgccatctttgggatgtccaactttgcctatgttaagagggaagttgggatcgatgacatgttcaactttgagacctttggcaacagcatgatctgcctattccaaattacaacctctgctggctgggatggattgctagcacccattctcaacagtaagccacccgactgtgaccctaataaagttaaccctggaagctcagttaagggagactgtgggaacccatctgttggaattttcttttttgtcagttacatcatcatatccttcctggttgtggtgaacatgtacatcgcggtcatcctggagaacttcagtgttgctactgaagaaagtgcagagcctctgagtgaggatgactttgagatgttctatgaggtttgggagaagtttgatcccgatgcaactcagttcatggaatttgaaaaattatctcagtttgcagctgcgcttgaaccgcctctcaatctgccacaaccaaacaaactccagctcattgccatggatttgcccatggtgagtggtgaccggatccactgtcttgatatcttatttgcttttacaaagcgggttctaggagagagtggagagatggatgctctacgaatacagatggaagagcgattcatggcttccaatccttccaaggtctcctatcagccaatcactactactttaaaacgaaaacaagaggaagtatctgctgtcattattcagcgtgcttacagacgccaccttttaaagcgaactgtaaaacaagcttcctttacgtacaataaaaacaaaatcaaaggtggggctaatcttcttataaaagaagacatgataattgacagaataaatgaaaactctattacagaaaaaactgatctgaccatgtccactgcagcttgtccaccttcctatgaccgggtgacaaagccaattgtggaaaaacatgagcaagaaggcaaagatgaaaaagccaaagggaaataa

SEQ ID NO: 13 - protein, 2009 aa, synthetic construct; identical to SEQ ID NO: 11

SEQ ID NO: 14 - DNA, 6036 bp, synthetic construct
  source 1..6036: mol_type = other DNA
gccaccatggagcagacagtcctggtcccacccggccctgattcattcaactttttcacaagagagagcctggcagctatcgagcgacggattgccgaggagaaggccaagaacccaaagcccgacaagaaggacgatgacgagaacggccctaagccaaattccgacctggaggccggcaagaatctgcccttcatctacggcgacatcccccctgagatggtgtctgagcccctggaggacctggacccctactatatcaacaagaagacctttatcgtgctgaataagggcaaggccatctttagattctccgccacatctgccctgtatatcctgaccccattcaaccccctgcggaagatcgccatcaagatcctggtgcacagcctgttttccatgctgatcatgtgcacaatcctgaccaattgcgtgttcatgacaatgagcaacccacccgactggaccaagaatgtggagtacaccttcaccggcatctataccttcgagtccctgatcaagatcatcgccaggggcttctgcctggaggacttcaccttcctgagggacccctggaactggctggacttcaccgtgatcaccttcgcctacgtgaccgagtttgtggacctgggcaacgtgagcgccctgaggacattcagagtgctgcgggccctgaagaccatctctgtgatccctggcctgaagacaatcgtgggcgccctgatccagtccgtgaagaagctgtctgatgtgatgatcctgacagtgttttgcctgagcgtgttcgccctgatcggcctccagctgttcatgggcaacctgaggaataagtgtatccagtggcctccaaccaacgccagcctggaggagcactccatcgagaagaatatcacagtgaactacaatggcacactgatcaacgagacagtgttcgagttcgactggaagtcttatatccaggatagccgctatcactactttctggagggcttcctggacgcactgctgtgcggcaacagctccgatgcaggacagtgccctgagggctacatgtgcgtgaaggccggcaggaacccaaattatggctacacatcctttgacaccttctcttgggcctttctgagcctgttccgcctgatgacccaggatttctgggagaacctgtaccagctgacactgagggccgccggcaagacctatatgatcttcttcgtgctggtcatcttcctgggctccttctacctgatcaatctgatcctggccgtggtggctatggcctacgaggagcagaaccaggccacactggaggaggccgagcagaaggaggccgagttccagcagatgatcgagcagctgaagaaacagcaagaagccgctcagcaagctgccaccgccaccgcatctgagcacagcagagagccatccgccgccggccggctgtctgactctagctccgaggccagcaagctgtctagcaagtccgccaaggagaggagaaaccggcgcaagaagcgcaagcagaaggagcagagcggcggcgaggagaaggacgaggatgagtttcagaagtctgagagcgaggactccatcaggagaaagggctttaggttctctatcgagggcaacagactgacctacgagaagcggtattcctctccccaccagtccctgctgtctatcagaggcagcctgttctcccctcggcgcaattctcggaccagcctgttttccttcaggggcagagccaaggacgtgggctccgagaatgattttgccgatgacgagcactctacattcgaggacaacgagtctaggagagatagcctgtttgtgcctcggcgccacggcgagaggagaaactctaatctgagccagacctcccgcagctccaggatgctggccgtgttcccagccaacggcaagatgcacagcaccgtggactgcaatggagtggtgtccctggtcggaggaccttccgtgccaacatctcccgtgggccagctgctgcctgaagtgatcatcgataagccagccaccgatgacaacggcaccacaaccgagacagagatgcgcaagcggcgctctagctcctttcacgtgagcatggatttcctggaggacccttcccagaggcagagggcaatgtctatcgccagcatcctgacaaataccgtggaggagctggaggagagcaggcagaagtgccccccttgttggtacaagttttctaatatcttcctgatctgggactgctccccttattggctgaaggtgaagcacgtggtgaacctggtggtcatggaccccttcgtggacctggccatcacaatctgtatcgtgctgaataccctgtttatggctatggagcactacccaatgacagaccacttcaacaatgtgctgaccgtgggcaacctggtgtttacaggcatcttcaccgccgagatgtttctgaagatcatcgctatggacccctactattacttccaggagggctggaatatctttgatggcttcatcgtgaccctgtccctggtggagctgggcctggcaaacgtggagggcctgagcgtgctgagatcctttcggctgctgcgcgtgttcaagctggccaagtcttggccaaccctgaacatgctgatcaagatcatcggcaacagcgtgggcgccctgggcaatctgacactggtgctggccatcatcgtgtttatcttcgccgtggtgggcatgcagctgttcggcaagagctacaaggactgcgtgtgcaagatcgcatccgattgccagctgccaaggtggcacatgaacgacttctttcacagctttctgatcgtgttcagggtgctgtgcggcgagtggatcgagacaatgtgggattgcatggaggtggcaggacaggcaatgtgcctgacagtgttcatgatggtcatggtcatcggcaacctggtggtgctgaatctgtttctggccctgctgctgtctagcttctccgccgacaacctggcagcaaccgatgacgataatgagatgaacaacctccagatcgccgtggatagaatgcacaagggcgtggcctacgtgaagcggaaaatctacgagtttatccagcagagcttcatcagaaagcagaagatcctggacgagatcaagccactggacgatctgaacaataagaaggattcctgcatgtctaatcacaccgccgagatcggcaaggacctggattacctgaaggacgtgaacggcacaacctccggcatcggcacaggctcctctgtggagaagtacatcatcgacgagtctgattatatgagcttcatcaacaatccctccctgacagtgaccgtgcctatcgccgtgggcgagtctgactttgagaacctgaataccgaggacttcagctccgagtccgatctcgaggagtctaaggagaagctgaacgagtctagctcctctagcgagggcagcacagtggacatcggcgccccagtggaggagcagcctgtggtggagccagaggagacactggagcccgaggcctgctttaccgagggctgcgtgcagaggttcaagtgctgtcagatcaatgtggaggagggcaggggcaagcagtggtggaacctgaggagaacctgttttagaatcgtggagcacaactggtttgagacattcatcgtgtttatgatcctgctgtcctctggcgccctggccttcgaggacatctacatcgaccagcggaagacaatcaagaccatgctggagtacgccgacaaggtgtttacctatatcttcatcctggagatgctgctgaagtgggtggcctatggctaccagacatactttaccaacgcctggtgctggctggatttcctgatcgtggacgtgagcctggtgtccctgaccgccaatgccctgggctattctgagctgggcgccatcaagagcctgaggaccctgagagccctgcggcccctgcgcgccctgtccagattcgagggcatgagggtggtggtgaatgcactgctgggagccatccccagcatcatgaacgtgctgctggtgtgcctgatcttttggctgatcttctccatcatgggcgtgaatctgtttgccggcaagttctaccactgcatcaacacaaccacaggcgacagattcgacatcgaggacgtgaacaatcacaccgactgtctgaagctgatcgagcgcaacgagacagccaggtggaagaatgtgaaggtgaactttgataatgtgggcttcggctatctgtccctgctccaggtggccaccttcaagggctggatggacatcatgtacgccgccgtggatagccggaacgtggagctgcaacctaagtatgaggagtccctgtatatgtacctgtatttcgtgatctttatcatcttcggcagcttctttaccctgaatctgtttatcggcgtgatcatcgacaacttcaatcagcagaagaagaagtttggcggccaggacatcttcatgacagaggagcagaagaagtattacaacgccatgaagaagctgggctccaagaagccacagaagcccatccctagaccaggcaataagtttcagggcatggtgtttgacttcgtgacccggcaggtgttcgacatctctatcatgatcctgatctgcctgaacatggtgacaatgatggtggagacagacgatcagtctgagtacgtgaccacaatcctgagccgcatcaatctggtgtttatcgtgctgttcaccggcgagtgcgtgctgaagctgatcagcctgaggcactattactttacaatcggctggaacatctttgacttcgtggtggtcatcctgtctatcgtgggcatgttcctggccgagctgatcgagaagtattttgtgagccccaccctgttcagagtgatcagactggcaaggatcggacgcatcctgaggctgatcaagggcgccaagggcatcagaacactgctgtttgccctgatgatgagcctgccagccctgttcaacatcggcctgctgctgttcctggtcatgttcatctacgccatctttggcatgtccaatttcgcctatgtgaagcgggaagtgggcatcgacgatatgtttaacttcgagacattcggcaacagcatgatctgcctgttccagatcaccacaagcgccggctgggacggcctgctggcaccaatcctgaactctaagccacccgactgcgatcccaacaaggtcaatcctggcagctccgtgaagggcgattgtggcaatcctagcgtgggcatcttctttttcgtgagctacatcatcatctcctttctggtggtggtgaacatgtatatcgccgtgatcctggagaatttctccgtggcaaccgaggagtctgccgagccactgagcgaggacgattttgagatgttctacgaagtgtgggagaagtttgaccctgatgccacacagtttatggagttcgagaagctgagccagttcgccgccgccctggagcctccactgaacctgccccagcctaacaagctccagctgatcgctatggacctgcctatggtgtctggcgacagaatccactgcctggacatcctgtttgccttcaccaagcgggtgctgggagagagcggagagatggatgcactgcgcatccagatggaggagaggttcatggcctctaacccaagcaaggtgtcctaccagcccatcaccacaaccctgaagcggaagcaggaggaggtgtccgccgtgatcatccagagggcataccggcgccacctgctgaagaggacagtgaagcaggcctctttcacctataacaagaataagatcaagggcggcgccaacctgctgatcaaggaggatatgatcatcgacagaatcaacgagaatagcatcacagagaagaccgacctgacaatgtccaccgccgcctgtcccccttcttatgatcgggtgactaaacctattgtggaaaaacacgagcaggagggaaaggatgagaaggcaaagggaaagtga

SEQ ID NO: 15 - protein, 1998 aa, Homo sapiens
MEQTVLVPPGPDSFNFFTRESLAAIERRIAEEKAKNPKPDKKDDDENGPKPNSDLEAGKNLPFIYGDIPPEMVSEPLEDLDPYYINKKTFIVLNKGKAIFRFSATSALYILTPFNPLRKIAIKILVHSLFSMLIMCTILTNCVFMTMSNPPDWTKNVEYTFTGIYTFESLIKIIARGFCLEDFTFLRDPWNWLDFTVITFAYVTEFVDLGNVSALRTFRVLRALKTISVIPGLKTIVGALIQSVKKLSDVMILTVFCLSVFALIGLQLFMGNLRNKCIQWPPTNASLEEHSIEKNITVNYNGTLINETVFEFDWKSYIQDSRYHYFLEGFLDALLCGNSSDAGQCPEGYMCVKAGRNPNYGYTSFDTFSWAFLSLFRLMTQDFWENLYQLTLRAAGKTYMIFFVLVIFLGSFYLINLILAVVAMAYEEQNQATLEEAEQKEAEFQQMIEQLKKQQEAAQQAATATASEHSREPSAAGRLSDSSSEASKLSSKSAKERRNRRKKRKQKEQSGGEEKDEDEFQKSESEDSIRRKGFRFSIEGNRLTYEKRYSSPHQSLLSIRGSLFSPRRNSRTSLFSFRGRAKDVGSENDFADDEHSTFEDNESRRDSLFVPRRHGERRNSNLSQTSRSSRMLAVFPANGKMHSTVDCNGVVSLVGGPSVPTSPVGQLLPEGTTTETEMRKRRSSSFHVSMDFLEDPSQRQRAMSIASILTNTVEELEESRQKCPPCWYKFSNIFLIWDCSPYWLKVKHVVNLVVMDPFVDLAITICIVLNTLFMAMEHYPMTDHFNNVLTVGNLVFTGIFTAEMFLKIIAMDPYYYFQEGWNIFDGFIVTLSLVELGLANVEGLSVLRSFRLLRVFKLAKSWPTLNMLIKIIGNSVGALGNLTLVLAIIVFIFAVVGMQLFGKSYKDCVCKIASDCQLPRWHMNDFFHSFLIVFRVLCGEWIETMWDCMEVAGQAMCLTVFMMVMVIGNLVVLNLFLALLLSSFSADNLAATDDDNEMNNLQIAVDRMHKGVAYVKRKIYEFIQQSFIRKQKILDEIKPLDDLNNKKDSCMSNHTAEIGKDLDYLKDVNGTTSGIGTGSSVEKYIIDESDYMSFINNPSLTVTVPIAVGESDFENLNTEDFSSESDLEESKEKLNESSSSSEGSTVDIGAPVEEQPVVEPEETLEPEACFTEGCVQRFKCCQINVEEGRGKQWWNLRRTCFRIVEHNWFETFIVFMILLSSGALAFEDIYIDQRKTIKTMLEYADKVFTYIFILEMLLKWVAYGYQTYFTNAWCWLDFLIVDVSLVSLTANALGYSELGAIKSLRTLRALRPLRALSRFEGMRVVVNALLGAIPSIMNVLLVCLIFWLIFSIMGVNLFAGKFYHCINTTTGDRFDIEDVNNHTDCLKLIERNETARWKNVKVNFDNVGFGYLSLLQVATFKGWMDIMYAAVDSRNVELQPKYEESLYMYLYFVIFIIFGSFFTLNLFIGVIIDNFNQQKKKFGGQDIFMTEEQKKYYNAMKKLGSKKPQKPIPRPGNKFQGMVFDFVTRQVFDISIMILICLNMVTMMVETDDQSEYVTTILSRINLVFIVLFTGECVLKLISLRHYYFTIGWNIFDFVVVILSIVGMFLAELIEKYFVSPTLFRVIRLARIGRILRLIKGAKGIRTLLFALMMSLPALFNIGLLLFLVMFIYAIFGMSNFAYVKREVGIDDMFNFETFGNSMICLFQITTSAGWDGLLAPILNSKPPDCDPNKVNPGSSVKGDCGNPSVGIFFFVSYIIISFLVVVNMYIAVILENFSVATEESAEPLSEDDFEMFYEVWEKFDPDATQFMEFEKLSQFAAALEPPLNLPQPNKLQLIAMDLPMVSGDRIHCLDILFAFTKRVLGESGEMDALRIQMEERFMASNPSKVSYQPITTTLKRKQEEVSAVIIQRAYRRHLLKRTVKQASFTYNKNKIKGGANLLIKEDMIIDRINENSITEKTDLTMSTAACPPSYDRVTKPIVEKHEQEGKDEKAKGK

SEQ ID NO: 16 - protein, 1998 aa, Homo sapiens; identical to SEQ ID NO: 15

(19) United States
(12) Patent Application Publication
(10) Pub. No.: US 2025/0262322 A1
(43) Pub. Date: Aug. 21, 2025

(54) A VECTOR, COMPOSITION, AND METHOD TO PROVIDE EXOGENOUS NAV1.1 ACTIVITY VIA CAV-2-MEDIATED DELIVERY OF AN SCN1A EXPRESSION CASSETTE

(71) Applicants: CENTRE NATIONAL DE LA RECHERCHE SCIENTIFIQUE, PARIS (FR); FUNDACION PARA INVESTIGACION MEDICA APLICADA, Pamplona (ES); Université de Montpellier, MONTPELLIER (FR)

(72) Inventors: Eric KREMER, Montpellier (FR); Bertrand BEUCHER, Montpellier (FR); Iria GONZALEZ DOPESO-REYES, Montpellier (FR); Ruben HERNANDEZ ALCOCEBA, Pamplona (ES); Ana Lourdes RICOBAZARA ABARQUERO, Pamplona (ES); Saja FADILA, Tel Aviv (IL); Moran RUBINSTEIN, Tel Aviv (IL)

(21) Appl. No.: 18/856,604

(22) PCT Filed: Apr. 11, 2023
(86) PCT No.: PCT/IB2023/053703

§ 371 Date: Oct. 11, 2024

(30) Foreign Application Priority Data
Apr. 11, 2022 (GB) 2205299.7

(51) Int. Cl. A61K 48/00; A61K 38/17; C07K 14/705; C12N 15/85

(52) U.S. Cl. CPC A61K 48/005; A61K 38/177; C07K 14/705; C12N 15/85

(57) ABSTRACT

A method for use in the treatment of diseases affecting the brain by providing exogenous Nav1.1 via CAV-2 vector-mediated delivery of a copy of SCNIA ORF into central nervous system (CNS) neurons. The method includes injection of the viral vector into the hippocampus and/or thalamus. Transcriptional control of the SCNIA expression cassette can be provided by specific promoters, such as NSE, to achieve targeted neuronal expression. This strategy is suitable as a therapeutic approach regardless of the underling SCNIA mutation. Moreover, because this approach compensates for the loss of function of Nav1.1, it can potentially ameliorate Dravet Syndrome, epileptic and non-epileptic comorbidities thereof, epilepsy, Alzheñner's, and other Nav1.1-associated pathological conditions.

### CROSS-REFERENCE TO RELATED APPLICATIONS

The present application is a filing under 35 U.S.C. 371 as the National Stage of International Application No. PCT/IB2023/053703, filed Apr. 11, 2023, entitled “A VECTOR, COMPOSITION, AND METHOD TO PROVIDE EXOGENOUS NAV1.1 ACTIVITY VIA CAV-2-MEDIATED DELIVERY OF AN SCN1A EXPRESSION CASSETTE,” which claims priority to United Kingdom Application No. 2205299.7 filed with the Intellectual Property Office of the United Kingdom on Apr. 11, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4692-19100 BNT241911USPC Sequence listing.xml; created on Oct. 4, 2024; and having a file size of 67.5 KB.

The information in the Sequence Listing is incorporated herein in its entirety for all purposes.

### BACKGROUND

Dravet syndrome (DS) is a rare and severe genetic neurological form of developmental epileptic encephalopathy (DEE). Children with DS appear to develop normally during the first six months of life, but subsequently start to exhibit febrile seizures. During the following months, recurrent refractory spontaneous seizures become increasingly more frequent and global developmental delays begin. Disease progression includes intellectual disability, behavioral and movement disorders, and a high mortality rate. It is one of the better-known rare diseases where epilepsy is one of the main symptoms. At early school years, during the chronic phase of the disease, the frequency of seizures declines, but the non-epileptic comorbidities persist.

Previously known as severe myoclonic epilepsy of infancy, DS is caused, in the majority of cases, by loss-of-function (LoF) mutations in one of two copies of SCN1A. The vast majority of cases are caused by de novo mutations in one copy of SCN1A, causing LoF and therefore a haploinsufficiency in the activity of the voltage-gated sodium channel, Nav1.1. As a result of these mutations, patients with DS fail to produce sufficient levels of functional Nav1.1 sodium channel, preventing inhibitory neurons from firing properly. The consequence is an imbalance between brain excitation and inhibition that results in refractory epilepsy, intellectual disability, and behavioral and movement disorders.

The first generation of therapies for the treatment of DS focused primarily on addressing symptoms, for example, by improving seizure control through use of anti-epileptic medications. This includes compounds against traditional epilepsy targets, such as GABA receptors, as well as novel first-in-class therapeutics that target new pathways not previously used in epilepsy. In recent years DS has received significant attention from the pharmaceutical industry. Following the approvals of Diacomit (stiripentol) from Biocodex and Epidiolex/Epidyolex (cannabidiol oral formulation) from GW Pharmaceuticals, Fintepla (fenfluramine) by Zogenix has recently become the third drug approved by both the FDA and the EMA for the treatment of seizures in DS.

Importantly, sodium channel blockers, which are often a first-line medication for the treatment of epilepsy, are contraindicated in DS and can aggravate the disease severity. Most patients must take combinations of three or more anti-epileptic drugs, most commonly valproate, clobazam, stiripentol, and topiramate and levetiracetam. None of these drugs alone achieve complete seizure suppression and only a minority (about 10%) of the patients become seizure-free.

There is also an emergent class of second-generation therapeutics that follow the step of the most advanced cannabidiol formulation, Epidiolex, and the most advanced serotonergic drug, Fintepla. Most of these compounds have the potential to treat other forms of epilepsy as well.

As the DS pipeline becomes more mature and competition increases, focus is shifting towards less symptomatic treatments and more disease-targeting therapeutics. A third generation of treatments aims to restore the abnormal Nav1.1 channel function or expression levels. There are currently several types of disease-targeting gene therapy approaches in development.

So called, “read-through therapies” such as the types produced by PTC Therapeutics and Tevard Biosciences enable the ribosome to move past a nonsense mutation to complete a functional protein. Nonsense mutations are those that introduce a premature stop codon into a gene sequence, preventing the cell from producing a complete protein. Despite its promise, an investigator-initiated study evaluated the safety and efficacy of ataluren for treating DS caused by nonsense mutations, and it indicated that ataluren failed to show efficacy.

Tevard Biosciences announced it was developing two novel tRNA-based gene therapy platforms for the treatment of DS and other rare diseases caused by haploinsufficiency and/or nonsense mutations that were not amenable to traditional approaches of gene therapy. Tevard's approach focused on mRNA stabilization and nonsense codon suppression (or read-though).

Stoke Therapeutics is developing the first disease-modifying treatment for DS, an antisense oligonucleotide (ASO) therapy targeting SCN1A pre-mRNA maturation that upregulates Nav1.1 protein expression in rodents, human cell lines, and non-human primates. The lead candidate uses Stoke's TANGO (Targeted Augmentation of Nuclear Gene Output) technology to target RNA splicing and increase protein expression in diseases caused by haploinsufficiency. The program, named STK-001, follows a similar approach to Spinraza and will be administered via intrathecal injection into the spinal fluid.

In mice with Scn1a haploinsufficiency, a single intracerebroventricular dose of a lead ASO increased production of Nav1.1 and met the two main preclinical endpoints in DS mouse models, which are seizure reduction and animal survival. Stoke has also shown biodistribution and target engagement of STK-001 in non-human primates (AES 2019 meeting) and dosed the first patient in a Phase 1a/2b study in August 2020.

Encoded Therapeutics is developing an adeno-associated virus (AAV)-based gene therapy approach to increasing SCN1A transcription and rescuing haploinsufficiency in patients with DS. The therapy, named ETX101, has the potential to become the second disease-modifying therapy to reach clinical trials for the treatment of DS after Stoke Therapeutics' ASO lead program. ETX101 was granted Orphan Drug Designation and Rare Pediatric Disease Designation by the FDA for the treatment of SCN1A+ DS.

The SCN1A ORF is poorly amenable for AAV-based gene therapy due tore the limited cloning capacity of <5,000 nt. Encoded Therapeutics attempted to overcome the limitations of AAV viral gene therapy by expressing an engineered transcription factor to upregulate Scn1a expression in these cells, and presented preclinical data from their program in 2019 showing GABAergic cell-specific expression of SCN1A mRNA and Nav1.1 channel in a mouse model of DS, leading to a reduction in seizures and mortality. In December 2020, the company also presented data on biodistribution of ETX101 in non-human primates.

The present disclosure relates to a method of providing exogenous Nav1.1 via vector delivery of an SCN1A expression cassette into the central nervous system (CNS). This strategy is suitable as a therapeutic approach regardless of the underling SCN1A mutation. Moreover, because this treatment compensates for the LoF of Nav1.1, it can potentially ameliorate DS epileptic and non-epileptic comorbidities as well as Alzheimer's and other Nav1.1 related pathological conditions. The minimum size of the expression cassette is ˜7 kbp. To overcome the “size” obstacle, previous gene therapy strategies for DS included (i) expression enhancement of the endogenous Scn1a via transcriptional activation (Colasante et al., 2019; Han et al., 2020; Yamagata et al., 2020); (ii) overexpression of the Scn1b auxiliary subunit for an increase of channel complex efficacy; and, (iii) antisense oligonucleotide-mediated downregulation of SCN8A.

While previous approaches to treating DS have shown potential improvements in “DS mice”, therapeutic potential in these approaches was only shown in cases of mild DS when administered soon after birth, during the asymptomatic, pre-epileptic stage. In essence, these approaches did not distinguish between models having a severe versus mild symptoms. Clinical efficacy in cases of severe condition of DS was not established. Moreover, as clinical diagnoses are rarely confirmed prior to progression to recurrent spontaneous seizures, therapies that prevent the onset of severe intractable seizures and sudden unexpected death in epilepsy (SUDEP) are critically needed.

In 2020, there was a transition in clinical trials for DS with the initiation of the first clinical trial of a therapy specifically designed to increase Nav1.1 activity via STK-001 by Stoke Therapeutics. There are additional genetic and potentially disease-modifying treatments in early and late preclinical development, including an AAV-based gene therapy.

In contrast to AAV, adenovirus (AdV) vectors can harbor up to 36 kbp of exogenous sequence. Similar to AAV vectors, some AdV types (e.g., E1-deleted and helper-dependent CAV-2 vectors) can generate long-term transgene expression in rodents and primate brain. Moreover, a human AdV (HAdVs) harboring an SCN1A expression cassette injected into the CNS of adolescent DS mice was well tolerated and reduced the epilepsy at the chronic stage. While AAV vectors have shown some promise in clinical trials, there remain safety concerns, including the extremely high doses needed and the tendency of AAV vectors to integrate into the genome of transduced cells. Like HAdV-based vector, most AAV vectors are also readily taken up by non-neuronal cells, including those that present antigens to immune cells. Moreover, most potential patients will have cross-reactive, memory T cells that recognize conserved AAV epitopes. Thus, the use of AAV and HAdV as a neuronal gene therapy vector could pose a risk. In addition, recent evidence demonstrated that AAV vectors are not as safe as originally thought, with reports of hepatotoxic, haematological toxicity, neurological safety issues and cancer concerns following AAV-mediated gene therapy, leading to premature termination of two studies (Mullard, 2021).

Therefore, a gene transfer vector is needed for preferential neuronal-uptake by a vector capable of harboring large expression cassettes for the delivery of SCN1A to the brain.

### SUMMARY

The vast majority of DS cases are caused by de novo mutations in one copy of SCN1A, causing LoF and, therefore, a haploinsufficiency in the activity of voltage-gated sodium channel, Nav1.1. Thus, a strategy in which exogenous Nav1.1 channels are provided into CNS neurons, can be used as therapeutic approach that will be suitable-regardless of the underling SCN1A mutation. Moreover, as this treatment compensates for the LoF of Nav1.1, it can potentially ameliorate epileptic and non-epileptic comorbidities as well as Alzheimer's and other Nav1.1 association pathological conditions.

One therapeutic approach is gene therapy, where one provides exogenous Nav1.1 via delivery of an SCN1A expression cassette. The gene therapy pipeline is intransigently linked to vector efficacy. Due to a paucity of vectors capable of harboring large expression cassettes, several rare diseases have little hope of being targets of gene replacement. Due to the complex physiopathology of Nav1.1 associated pathological conditions such as DS and Alzheimers, etiological approaches such as gene therapy have unique chances to obtain a global improvement in the life of these patients. However, several challenges for treatment include the large (6 kb) SCN1A ORF, which precludes efficient expression by some viral vectors, the possibility that supraphysiological Nav1.1 levels in non-neuronal cells could be harmful, infecting enough neurons in the pertinent regions, and providing long-term expression. The present disclosure relates to a gene transfer vector and method of treatment that will pave the way for clinical trials for DS, epilepsy, Alzheimer's, and other genetic diseases and pathological conditions associated with miscoded Nav1.1.

Due to the ˜6 kbp ORF of SCN1A, commonly used vectors, such as adeno-associated virus (AAV), are poorly suitable. In contrast to AAV, AdV vectors can harbor up to 36 kbp of exogenous sequence (Cots et al., 2013; del Rio et al., 2019; Parks et al., 1996). AdV-mediated transgene expression is also stable for at least 1 year in the brain of rodents (Soudais et al., 2004). Moreover, a human AdV harboring the SCN1A injected into the CNS of adolescent mice with DS was well tolerated and reduced the epilepsy at the chronic stage (Mora-Jimenez et al., 2021). However, HAdVs lack neuronal specificity, relied on a different non-neuronal specific promoter, CAG, and have only shown efficacy at ameliorating epilepsy and behavioral problems in older mice who have a less severe expression of DS.

Fatality in DS mice is ˜50-60%, with most deaths clustered in the 4th week of life. DS mice that survive beyond P28 are less likely to die and their seizure frequency is reduced. Previously, studies have been conducted on older mice at around 5 weeks, wherein their brains were injected with a HAdV harboring the SCN1A expression cassette. However, as the severe stage of DS corresponds to the 4th week of life, by treating adolescent mice at five weeks of age, these studies were preselecting for mice with milder conditions of DS. These studies do not relate to the reversion of DS at the severe stage of the disease when epilepsy is most severe and the occurrence of premature mortality is higher.

This disclosure relates to a canine adenovirus type 2 (CAV-2)-mediated SCN1A ORF delivery for use in the treatment of SCN1A mutation and Nav1.1 sodium channel miscoding as well as rescue from the severe stage of DS and prevention of premature death. For example, in certain embodiments, the present disclosure relates to CAV-2-mediated delivery of an SCN1A ORF expression cassette for use in the treatment of SCN1A mutation and Nav1.1 sodium channel miscoding. This could enable rescue from severe DS and prevention of premature death. It would also provide significant therapeutic value in the treatment of Alzheimer's and other Nav1.1 associated pathological conditions.

CAV-2 vectors offer notable advantages for gene transfer to the CNS. First, by preferentially using the coxsackievirus and adenovirus receptor (CAR) as an attachment molecule—whose expression is found primarily by neurons in the brain parenchyma, CAV-2-mediated gene transfer is delimited to neurons. Second, the 35 kbp cloning capacity offers the ability to include different promoter sequences, enabling control over expression in various sub-neuronal populations. Finally, with its robust retrograde axonal transport capability, CAV-2 vector injections can lead to expression across connected brain regions.

In some aspects, the present disclosure relates to a CAV-2 vector comprising a region of SCN1A ORF. The disclosure also relates to an engineered CAV-2 vector comprising a region of SCN1A ORF and further comprising an inducible promoter region for the controlled expression of the SCN1A ORF. The disclosure also relates to a CAV-2 vector. In certain embodiments, the CAV-2 vector can be enhanced for selective neuronal transgene expression. The disclosure also relates to a CAV-2 vector comprising a region of SCN1A ORF and can further comprise regions encoding for controlled (e.g., transcriptionally or translationally, or post-translational) SCN1A transcription, mRNA processing, protein stability or protein targeting.

In some aspects, the present disclosure relates to CAV-2-mediated CNS delivery of SCN1A ORF (CAV-2 SCN1A treatment) for rescue from DS and prevention of premature death. For example, the disclosure relates to CAV-2 SCN1A treatment for rescue from DS during the severe stage of the disease and prevention of premature death in mice with DS, and for correction of DS comorbidities. Importantly, exogenous Nav1.1 activity in CAV-2 SCN1A treated adolescent mice that survived to the chronic stage of DS, as well as in juvenile mice at the onset of severe epilepsy, improved survival of the mice, reduced the occurrence of spontaneous seizures and epileptic spike frequency, and increased the temperature threshold for febrile seizures. The present disclosure relates to the efficient delivery of an SCN1A expression cassette in excitatory and inhibitory neurons as a viable therapeutic approach for rescuing and treating DS. This disclosure also relates to treatment of DS and, specifically, treatment with CAV-2-mediated CNS delivery of SCN1A ORF in animal models with DS to correct DS comorbidities.

In some aspects, this disclosure relates to the design and use of a CAV-2 vector for the transfer of the SCN1A ORF to the CNS for the treatment of DS. By preferentially using the coxsackievirus and adenovirus receptor (CAR) as an attachment molecule—whose expression is found primarily by neurons in the brain parenchyma, CAV-2-mediated gene transfer is delimited to neurons. The cloning capacity of CAV-2 offers the ability to include different promoter sequences, enabling control over expression in various sub-neuronal populations. Additional features include its retrograde axonal transport capability, allowing CAV-2-mediated transduction to lead to expression across a plurality of connected brain regions.

In another aspect, the present disclosure relates to the inclusion of a suitable promoter to be used in an expression cassette for the SCN1A ORF, the expression cassette to be incorporated into the sequence of a CAV-2 vector. The promoter can be used for purposes of controlling expression levels of the SCN1A ORF. The promoter can also be selected based on the need to make modifications in the neuronal tropism of the vector. The CAV-2 sequence can also be engineered for purposes of selective neuronal and stable long-term expression.

In another aspect, the present disclosure relates to a method for producing an engineered CAV-2 SCN1A treatment vector. The production is carried out following methods developed at the collaborating center (CNRS, Montpellier), which rely on the use of a helper CAV-2 vectors. This helper vector replicates together with CAV-2 SCN1A treatment vector, however infectious helper vectors particles are not encapsidated because its packaging sequence, flanked by lox sequences, is eliminated by Cre recombinase.

In other embodiments, this disclosure relates to the efficient delivery of an SCN1A expression cassette in excitatory and/or inhibitory neurons as a viable therapeutic approach for DS. Thus, this disclosure relates to a strategy in which exogenous functional Nav1.1 channels are provided into CNS neurons via CAV-2 mediated delivery of SCN1A ORF, as a therapeutic approach that is suitable for children and adolescents-regardless of the underling SCN1A mutation. Moreover, this disclosure relates to a treatment that compensates for the LoF of Nav1.1, and can ameliorate DS epileptic and non-epileptic comorbidities and achieve rescue and reversion of DS as well as provide treatment and therapeutic methods for Nav1.1 associated pathological conditions.

Therefore, based on the foregoing and continuing description, the subject invention in its various embodiments may comprise one or more of the following features in any non-mutually-exclusive combination:
A vector for providing exogenous Nav1.1 sodium channels having a first nucleic acid sequence encoding at least one portion of a CAV-2 genome;
A vector for providing exogenous Nav1.1 sodium channels having a second nucleic acid sequence encoding a functional Nav1.1 sodium channel;
A vector for providing exogenous Nav1.1 sodium channels having a first regulatory sequence encoding a transcriptional control region,
A vector for providing exogenous Nav1.1 sodium channels wherein the transcriptional control region contains a promoter/enhancer/elongation factor sequence encoding at least one promoter/enhancer/elongation factor.
A vector for providing exogenous Nav1.1 sodium channels wherein a transcriptional control region contains a promoter/enhancer/elongation factor sequence encoding E2, E6, EF1, CAG, hSyn, Dlx5/6, and NSE, and preferably, NSE;
A vector for providing exogenous Nav1.1 sodium channels wherein a transcriptional control region contains a promoter/enhancer/elongation factor sequence encoding an endogenous SCN1A promoter;
A vector for providing exogenous Nav1.1 sodium channels wherein a transcriptional control region contains a promoter/enhancer/elongation factor sequence encoding an endogenous SCN1A enhancer;
A vector for providing exogenous Nav1.1 sodium channels wherein a transcriptional control region contains a promoter/enhancer/elongation factor sequence encoding an endogenous SCN1A transcription factor;
A vector for providing exogenous Nav1.1 sodium channels wherein a transcriptional control region contains a promoter/enhancer/elongation factor sequence encoding an endogenous SCN1A promoter;
A vector for providing exogenous Nav1.1 sodium channels wherein a transcriptional control region contains a promoter/enhancer/elongation factor sequence encoding an endogenous SCN1A enhancer;
A vector for providing exogenous Nav1.1 sodium channels wherein a transcriptional control region contains a promoter/enhancer/elongation factor sequence encoding E6;
A vector for providing exogenous Nav1.1 sodium channels wherein a transcriptional control region contains a promoter/enhancer/elongation factor sequence encoding EF1;
A vector for providing exogenous Nav1.1 sodium channels wherein a transcriptional control region contains a promoter/enhancer/elongation factor sequence encoding CAG;
A vector for providing exogenous Nav1.1 sodium channels wherein a transcriptional control region contains a promoter/enhancer/elongation factor sequence encoding hSyn;
A vector for providing exogenous Nav1.1 sodium channels wherein a transcriptional control region contains a promoter/enhancer/elongation factor sequence encoding Dlx5/6;
A vector for providing exogenous Nav1.1 sodium channels wherein a transcriptional control region contains a promoter/enhancer/elongation factor sequence encoding NSE;
A vector for providing exogenous Nav1.1 sodium channels wherein a second nucleic acid sequence is operably linked to the first regulatory sequence for controlled transcriptional expression of an SCN1A open reading frame (ORF) in mammalian cells;
A vector for providing exogenous Nav1.1 sodium channels wherein a second nucleic acid sequence is Sequence ID Number 12;
A vector for providing exogenous Nav1.1 sodium channels wherein a second nucleic acid sequence is Sequence ID Number 14;
A vector for providing exogenous Nav1.1 sodium channels wherein a second nucleic acid sequence encodes for Sequence ID Number 15;
A vector for providing exogenous Nav1.1 sodium channels wherein a second nucleic acid sequence encodes for Sequence ID Number 16;
A vector for providing exogenous Nav1.1 sodium channels wherein a second nucleic acid sequence is an engineered SCN1A ORF;
A vector for providing exogenous Nav1.1 sodium channels wherein a engineered SCN1A ORF is created by eliminating at least one short repeat sequence from a codon-modified SCN1A ORF;
A vector for providing exogenous Nav1.1 sodium channels wherein a second nucleic acid sequence is created by eliminating at least one short repeat sequence in an ORF encoding a transmembrane section of the functional Nav1.1 sodium channel;
A vector for providing exogenous Nav1.1 sodium channels wherein the vector is designed for use in the treatment of pathological conditions associated with SCN1A mutation;
A vector for providing exogenous Nav1.1 sodium channels having an NSE promoter;
A vector for providing exogenous Nav1.1 sodium channels wherein the vector is therapeutically active in individuals with SCN1A missense mutations or SCN1A truncation mutations;
A vector for providing exogenous Nav1.1 sodium channels wherein the vector is optimized for preferential expression of SCN1A ORF in neurons;
A vector for providing exogenous Nav1.1 sodium channels wherein the vector is therapeutically active in individuals with Dravet Syndrome;
A vector for providing exogenous Nav1.1 sodium channels wherein the vector is therapeutically active in individuals with Epilepsy;
A vector for providing exogenous Nav1.1 sodium channels wherein the vector is therapeutically active in individuals with Alzheimer's disease;
A vector for providing exogenous Nav1.1 sodium channels wherein the vector is therapeutically active in individuals with Autism Spectrum Disorder;
A vector for providing exogenous Nav1.1 sodium channels wherein the vector is therapeutically active in individuals with neuronal disfunction;
A vector for providing exogenous Nav1.1 sodium channels wherein the vector is therapeutically active in individuals with network disfunction;
A vector for providing exogenous Nav1.1 sodium channels wherein the vector is therapeutically active in individuals with a loss of function of endogenous Nav1.1;
A method for providing exogenous Nav1.1 channels via a vector including administering a therapeutically effective amount of any of the aforementioned vectors, including a vector for providing exogenous Nav1.1 sodium channels;
A method for providing exogenous Nav1.1 channels via a vector including wherein the vector is administered into a central nervous system of the mammal;
A method for providing exogenous Nav1.1 channels via a vector including wherein the vector is administered into at least one brain region;
A method for providing exogenous Nav1.1 channels via a vector including wherein the vector is administered into the hippocampus;
A method for providing exogenous Nav1.1 channels via a vector including wherein the vector is administered into the thalamus;
A method for providing exogenous Nav1.1 channels via a vector wherein the vector is administered prior to development of a chronic form of Dravet Syndrome or comorbidities thereof;
A method for providing exogenous Nav1.1 channels via a vector wherein the vector is administered to treat a chronic form of Dravet Syndrome or comorbidities thereof, · A method for providing exogenous Nav1.1 channels via a vector wherein the vector is administered to treat Dravet Syndrome or comorbidities thereof;
A method for providing exogenous Nav1.1 channels via a vector wherein the vector is administered to prevent the development of Dravet Syndrome or comorbidities thereof;
A method for providing exogenous Nav1.1 channels via a vector wherein the vector is administered to individuals with a missense mutation in SCN1A;
A method for providing exogenous Nav1.1 channels via a vector wherein the vector is administered to individuals with a nonsense mutation in SCN1A;
A method for providing exogenous Nav1.1 channels via a vector wherein the vector is administered to individuals with a truncation mutation in SCN1A;
A method for providing exogenous Nav1.1 channels via a vector wherein the vector is administered to individuals with a deletion mutation in SCN1A;
A method for providing exogenous Nav1.1 channels via a vector wherein the vector is administered to individuals with a frameshift mutation in SCN1A;
A method for providing exogenous Nav1.1 channels via a vector wherein the vector is administered to individuals with a splice site mutation in SCN1A;
A method for providing exogenous Nav1.1 channels via a vector wherein the vector is administered to individuals with a mutation in SCN1A;
A method for providing exogenous Nav1.1 channels via a vector wherein the vector is administered to individuals with a mutation in a promoter/enhancer/regulatory region of SCN1A;
A method for providing exogenous Nav1.1 channels via a vector wherein after administration of the vector, the method further includes inducing expression of the functional Nav1.1 sodium channel;
A method for providing exogenous Nav1.1 channels via a vector wherein the vector is administered to individuals with a missense mutation in SCN1A or truncation mutation in SCN1A;
A method for providing exogenous Nav1.1 channels via a vector wherein after administration of the vector, the method further comprises monitoring the mammal for desired clinical results, and if the desired clinical results are not obtained, administering a second therapeutically effective amount of the vector;
A method for providing exogenous Nav1.1 channels via a vector wherein the vector is administered via injection into a patient's brain;
A method for providing exogenous Nav1.1 channels via a vector including administering a therapeutically effective amount of seizure medication;
A method for providing exogenous Nav1.1 channels via a vector including administering a therapeutically effective amount of Alzheimer's medication;
A method for providing exogenous Nav1.1 channels via a vector including administering a therapeutically effective amount of epilepsy medication;
A method for providing exogenous Nav1.1 channels via a vector wherein therapeutically effective amount of the vector is administered for the treatment of Dravet Syndrome;
A method for providing exogenous Nav1.1 channels via a vector wherein therapeutically effective amount of the vector is administered for the treatment of Epilepsy;
A method for providing exogenous Nav1.1 channels via a vector wherein therapeutically effective amount of the vector is administered for the treatment of Alzheimer's;
A method for providing exogenous Nav1.1 channels via a vector to modify a brain neural network to reduce symptoms of a pathological condition.
A method for providing exogenous Nav1.1 channels via a vector to modify a brain neural network to reduce symptoms of Autism Spectrum Disorder (ASD).
A composition for providing exogenous Nav1.1 channels, the composition including any of the aforementioned vectors, or a vector for providing exogenous Nav1.1 sodium channels.

### BRIEF DESCRIPTION OF THE DRAWINGS

FIG. 1. Promoter-driven variations in CAV-2 vector transgene expression.

FIG. 1.1 Enhancer/promoter-driven variations in CAV-2 vector transgene expression.

FIG. 2. Functional expression of the SCN1A ORF.

FIG. 3. Survival of adolescent mice following injection of CAV-GFP or CAV-SCN1A.

FIG. 4. Hippocampal injection of CAV-SCN1A at the chronic stage of DS improves epilepsy and cognitive abilities.

FIG. 5. Sexual divergent effects following CAV-SCN1A injections into the hippocamps.

FIG. 6. Behavioral characterization of male and female mice.

FIG. 7. Hippocampal injection of CAV-SCN1A at the severe stage of DS ameliorates the epileptic phenotypes.

FIG. 8. Video monitoring of convulsive spontaneous seizures.

FIG. 9. Correction of background ECoG activity, and partial correction of cognitive functions following treatment with CAV-SCN1A.

FIG. 10. Relative expression of voltage-gated sodium channels expression.

FIG. 11. Thalamic injection of CAV-SCN1A ameliorates DS phenotypes in juvenile mice.

FIG. 12. Similar effects CAV-SCN1A injections into the thalamus of males and females DS mice.

FIG. 13. Combined thalamic and hippocampal injection of CAV-SCN1A in juvenile mice protects from thermally-induced seizures.

FIG. 14. Similar effects CAV-SCN1A injections into the thalamus and the hippocampus of males and females DS mice.

FIG. 15. The effect of CAV-SCN1A injections into the hippocampus and/or thalamus of juvenile mice.

FIG. 16. (A) Map of two CAV-2 vectors harboring either an mCitrine casette or a codon-modified SCN1A cDNA with fused HA tag, and the experimental design of the study in which they are bilaterally injected in the hippocampus of Alzheimer's mouse models. ITR: inverted terminal repeat. ψ: packaging signal. NSE (promoter): neuron-specific enolase. pA: polyadenylation signal. (B) Expression pattern of the vectors injected in the hippocampus. Second and third column are a higher magnification of, respectively, black and white frames of the first column. (C) Therapeutic effect of CAV-NSE-SCN1A-HA on working memory in the Y-maze test in 4 and 5-months-old J20 and 5XFAD mice. (D-E) Amyloid loads (white frames) analysis and quantification in the hippocampus and cortex of 9-months-old J20 mice.

### DETAILED SUMMARY

A detailed description of one or more embodiments of the claimed subject matter is provided below and herein along with accompanying figures that illustrate the principles of the claimed subject matter. The claimed subject matter is described in connection with such embodiments, but is not limited to any particular embodiment. It is to be understood that the claimed subject matter may be embodied in various forms, and encompasses numerous alternatives, modifications and equivalents. Therefore, specific details disclosed herein are not to be interpreted as limiting, but rather as a basis for the claims and as a representative basis for teaching one skilled in the art to employ the claimed subject matter in virtually any appropriately detailed system, structure, or manner. Numerous specific details are set forth in the following description in order to provide a thorough understanding of the present disclosure. These details are provided for the purpose of example and the claimed subject matter may be practiced according to the claims without some or all of these specific details. It is to be understood that other embodiments can be used and structural changes can be made without departing from the scope of the claimed subject matter. It should be understood that the various features and functionality described in one or more of the individual embodiments are not limited in their applicability to the particular embodiment with which they are described. They instead can be applied alone or in some combination to one or more of the other embodiments of the disclosure, whether or not such embodiments are described, and whether or not such features are presented as being a part of a described embodiment. For the purpose of clarity, technical material that is known in the technical fields related to the claimed subject matter has not been described in detail so that the claimed subject matter is not unnecessarily obscured.

All headings are for the convenience of the reader and should not be used to limit the meaning of the text that follows the heading, unless so specified.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range. For example, description of a percentage range such as at least 75 should be considered to have specifically disclosed sub-ranges such as from 75 to 100, from 80 to 100, from 85 to 100, from 95 to 100, from 99 to 100, etc., as well as individual numbers within that range, for example, 75, 76, 77, 78, 79, etc.

### Transcriptionally-Targeted Transgene Expression

Currently, DS is thought to be caused by inhibitory neuron dysfunction. It has also been postulated that reduced Nav1.1 activity in excitatory neurons may also be associated with DS comorbidities. Moreover, hippocampal dysfunction likely plays a key role in DS pathophysiology.

### Enhancer/Promoter-Driven Variations in CAV-2 Vector Expression

Assays were initiated to determine whether expression could be selectively targeted in different neuronal populations in the hippocampus and afferent regions. Because selective targeting of infection of various neuronal subtypes is not yet technically feasible, transcriptional control of the expression cassette was chosen to achieve targeted neuronal expression. For this purpose, CAV-2 vectors containing a fluorescent reporter driven by i) a strong non-specific CAG promoter (cytomegalovirus enhancer, chicken b-actin promoter and rabbit b-globin splice acceptor site) that drives expression in all vector-transduced cells; ii) a human synapsin promoter (hSyn), which drives transgene expression in most neuron populations; iii) a neuron-specific enolase (NSE) promoter, which drives moderate levels of transgene expression in excitatory and inhibitory neurons; iv) a Dlx5/6 promoter (Dlx5 and Dlx6 encode two homeobox transcription factors expressed by developing and mature GABAergic interneurons), which, when incorporated into a viral vector, can lead to preferential expression in inhibitory neurons; the Scn1a enhancers E2 and E6.

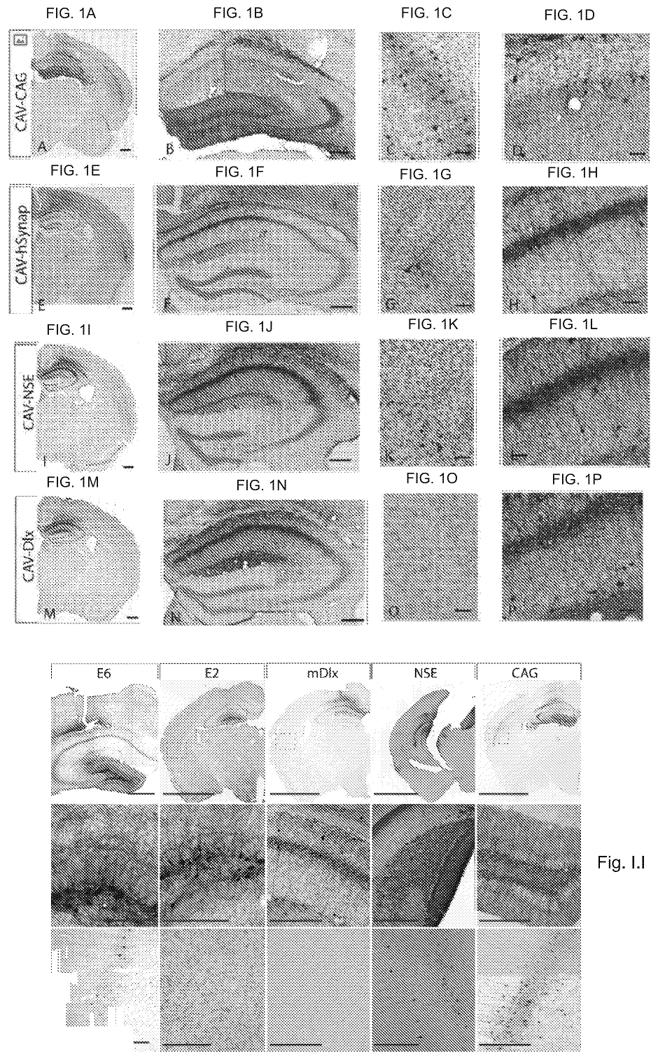

Following bilateral vector injection into the hippocampus of adolescent mice (5-6-week-old), the expression pattern varied depending on the promoter. As expected, the CAG promoter generated robust and widespread transgene expression at the site of injection and, due to the retrograde transport of CAV-2, also in excitatory neurons from the subiculum and multiple neocortical layers that project into the hippocampus (FIGS. 1A-D). Although at lower levels, the hSyn promoter also led to widespread transgene expression at the site of injection and afferent regions (FIG. 1 E-H). The NSE promoter resulted in an intermediate expression level in excitatory and inhibitory neurons around the injection sites (with a ratio of 80-90% excitatory and 10-20% inhibitory), as well as additional hippocampal projecting regions in the cortex and thalamus (FIG. 1 I-L). Conversely, using the Dlx5/6 promoter, a localized transgene expression principally in hippocampal interneurons was observed (FIG. 1 M-P). Based on the expression patterns in excitatory and inhibitory neurons, biodistribution, and the likely need for moderate levels of Nav1.1, pan-neuronal expression mediated by the NSE promoter was chosen to drive SCN1A expression using CAV-2 vectors.

FIG. 1 shows promoter-driven variations in the expression patterns and biodistribution of CAV-2 vectors containing a fluorescent reporter in excitatory and inhibitory neurons following bilateral vector injection into the hippocampus of adolescent mice of 5-6 weeks of age. FIGS. 1A-D show CAG promoter-driven variations. FIG. 1 E-H show hSynap promoter-driven variations. FIG. 1 I-L shows NSE promoter-driven variations. FIG. 1 M-P show Dlx5/6 promoter-driven variations. FIG. 1.1 shows enhancer/promoter driven variations as follows: 1^{st} column shows E6 enhancer from SCN1A; 2^{nd} column shows E2 enhancer from SCN1A; 3^{rd} column shows Dlx5/6; 4^{th} column shows NSE; and 5^{th} column shows CAG.

### CAV-SCN1A-Mediated Nav1.1 Activity

In addition to its size, the native SCN1A ORF is prone to rearrangement when subcloned into a plasmid and propagated in E. coli. However, codon modification can enhance its stability and, therefore, facilitate the creation of vectors. To further increase the stability of the ORF, short (10-15 bp) repeat sequences were eliminated from an engineered SCN1A. The majority of the short repeats were in the sequences coding for the transmembrane sections of Nav1.1.

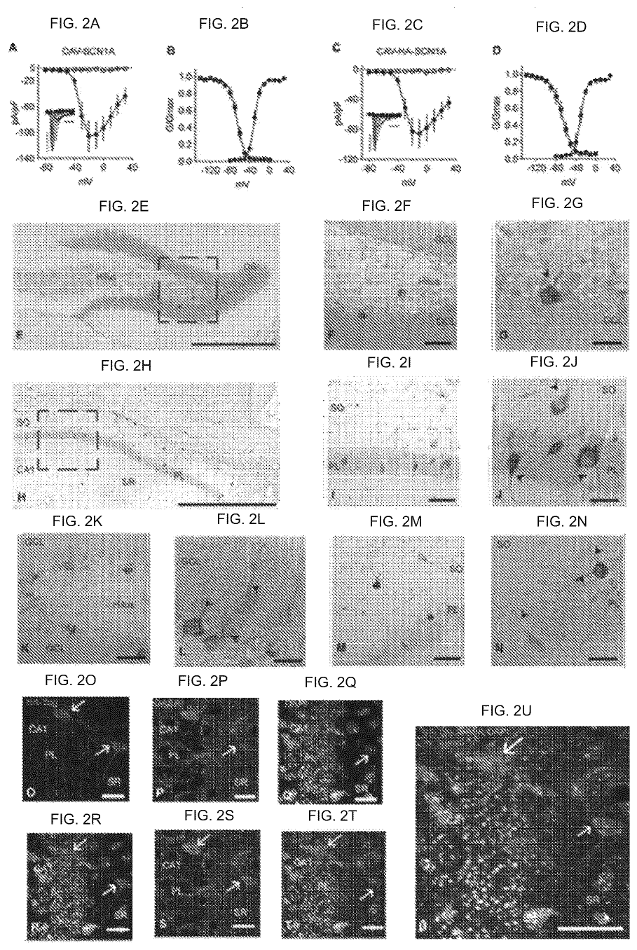

Detection of exogenous Nav1.1 immunoreactivity can be problematic in a brain with endogenous levels. Therefore, to follow CAV-SCN1A-mediated Nav1.1 expression, a C-terminal hemagglutinin (HA) tag was also added. CAV-SCN1A was generated and purified as previously described. To determine if functional Nav1.1 channels were generated, CAV-SCN1A was incubated with DK cells. Robust voltage-gated sodium currents with the biophysical properties that are characteristic of Nav1.1 were recorded (FIGS. 2A, B). Of note, the HA tag did not have a notable effect on the biophysical properties of the channel (FIG. 2 C, D). When injected into the hippocampus of adolescent mice, HA-immunoreactivity was readily detected along the dendrites and soma of excitatory neurons in the hippocampus as well as the dendrites, soma, and axon initial segment of inhibitory neurons.

### Functional Expression of the SCN1A Transgene

FIG. 2. illustrates the functional expression of the SCN1A transgene. With respect to FIGS. 2A-B, the voltage current relationship (A) and the biophysical properties (B) of sodium currents following expression of CAV-SCN1A in DK cells is shown. The half voltage of activation was-30.4±0.74 mV; the half voltage of inactivation was −65.73±0.94 mV. FIG. 2 C-D show the voltage current relationship and the biophysical properties of sodium currents in DK cells following expression of CAV-HA-SCN1A in DK cells. The half voltage of activation was-30.6±1.2 mV and half voltage of inactivation −67.8±1.8 mV. Inset in FIGS. 2A and C: a representative sodium current traces, calibrators: 500 pA, 2 ms. The empty symbols depict the current recorded from DK cells infected with the control CAV-GFP control vector (n=10), closed symbols depict CAV-SCN1A (n=9) or CAV-HA-SCN1A (n=7), as indicated.

As seen in FIG. 2 E-U, CAV-HA-SCN1A. 1×10⁹ physical particles were injected bilaterally into the hippocampus of adult mice. In FIG. 2 E-N, background staining is cresyl violet. HA expression is shown by immunohistology DAB staining (dark brown). Approximately 1,000 HA-immunoreactive cells/mouse, with neuronal morphology could be readily identified. FIG. 2 E and H show HA immunoreactivity in the hippocampus. Scale bar 500 μm. FIG. 2 F and I show magnification of the black box in FIG. 2 E and H, and additional examples FIG. 2 K and M. Scale bar 50 μm. FIG. 2 G, J, L, and N show magnification of the red box. Scale bar 20 μm. Figs. O-U show immunofluorescence in a mouse injected with CAV-HA-SCN1A. FIG. 20 shows HA immunoreactivity (IR). FIG. 2P shows GABA IR. FIG. 2Q shows DAPI IR and merge (FIG. 2 R-U). Scale bars 20 μm.

### CAV-SCN1A Injections Revert Epileptic Phenotypes in Adolescent DS Mice

There are multiple mouse models of DS that faithfully reproduce the hallmarks of DS pathology. These “DS mice” display age-dependent progression of the severity of the epilepsy with spontaneous seizures that begin around postnatal day (P) 18, and premature death that peaks during the 4th week of life. DS mice that survive this severe stage enter a chronic stage, in which the frequency of spontaneous convulsive seizures and mortality is reduced. Of note, genetic background dramatically affects the severity of the epileptic phenotype in DS mice.

Here, one of the most demanding models of DS was used, harboring a missense mutation (SCN1A^{A1783V/WT}) on the C57BL/6J background. In this model, all of the mice experience spontaneous seizures, with an overall mortality rate of over 50%. This mutation was shown to cause LoF of Nav1.1, recapitulating the characteristic neuronal alterations of DS. However, unlike other models with SCN1A truncation mutations, the A1783V missense mutation does not affect the overall SCN1A mRNA or total Nav1.1 levels.

Due to the retrograde transport of CAV-2 vectors, it was postulated that bilateral injections into the hippocampi would be sufficient to improve the epilepsy in adolescent SCN1A^{A1783V/WT} mice. To that end, bilateral injections of CAV-SCN1A or CAV-GFP were performed in healthy (WT) and SCN1A^{A1783V/WT} adolescent mice (5-weeks old). As noted above, after P30, during the chronic stage of the disease, such as in those of 5-week-old adolescent mice, the prevalence of sudden unexpected death in epilepsy (SUDEP) is low (Fadila et al., 2020; Ogiwara et al., 2013; Ricobaraza et al., 2019; Yu et al., 2006). Inasmuch, within this cohort of mock CAV-GFP injected and treated mice, only one mock-treated SCN1A^{A1783V/WT} mouse died.

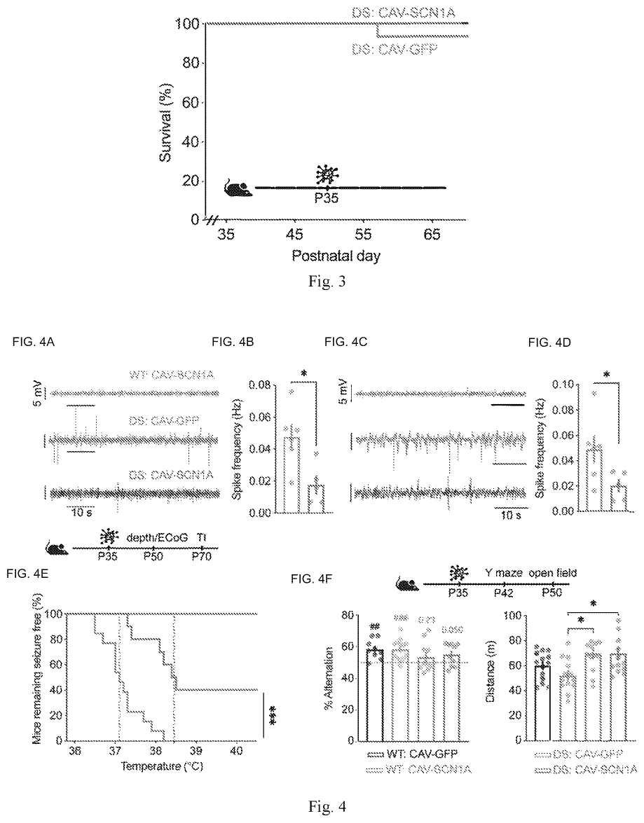

As shown in FIG. 3, no death occurred in WT mice injected with either CAV-GFP or CAV-SCN1A or DS mice injected with CAV-SCN1A. Premature death in DS mice mostly occurs at the 4th week of life. As shown in FIG. 3, the ability of CAV-SCN1A to ameliorate the epileptic phenotypes in a subset of mice that survived to the fifth week of life was tested. Only one DS mouse died prematurely (treated with CAV-GFP). WT: CAV-GFP n=11; WT: CAV-SCN1A n=12; DS: CAV-GFP n=9; DS: CAV-SCN1A n=12.

With minimal potential to effect survival, electrocorticography was used to characterize the impact of CAV-SCN1A on aberrant neuronal activity. Two weeks post-injection, depth electrodes were implanted in the hippocampus and intracranial electrodes were implanted in the somatosensory cortex for electrocorticography (ECOG) recordings. Initially, these recordings showed that CAV-SCN1A injections in WT mice did not induce aberrant neuronal activity (FIG. 4 A, C). Moreover, bilateral hippocampi CAV-SCN1A injections in SCN1A^{A1783V/WT} mice reduced, but did not completely eliminate, the occurrence of epileptic spike (FIGS. 4A-D). Another hallmark of DS is the sensitivity to thermally-induced seizure. Effective anti-seizure medications, which are used to treat patients with DS, can elevate the threshold for thermally-induced seizures in DS mice. Strikingly, ˜40% of the CAV-SCN1A-treated DS cohort showed complete protection from seizures up to 40.5° C. (FIG. 4E). Moreover, CAV-SCN1A injections in DS mice that exhibited seizures also demonstrated reduced susceptibility and elevated temperature threshold (FIG. 4E).

The present disclosure demonstrates the efficacy of CAV-SCN1A treatment to improve the clinical condition an outcome of epileptic phenotypes in SCN1A^{A1783V/WT} mice during the chronic stage of DS.

CAV-SCN1A does not Alter the Behavior of WT Mice

Next, the Y maze spontaneous alternation test, which assesses working memory based on the natural curiosity of mice and their tendency to explore novel environments, and the open field test, which monitors activity in a novel environment, were used to examine the effect of CAV-SCN1A injections in adolescent WT and DS mice. Significant differences between the groups were not detected (FIG. 4F). However, if the alteration level was random, it was found that WT mice injected with either CAV-GFP or CAV-SCN1A exhibited alteration above chance level. Conversely, SCN1A^{A1783V/WT} mice injected with CAV-GFP demonstrated random exploration of the maze (FIG. 4F), consistent with deficit in their working memory. Notably, DS mice injected with CAV-SCN1A exhibited exploration that was not random, and at the edge of the statistical significance (FIG. 4F), suggestive of some improvement of their cognitive abilities. In the open field test, CAV-SCN1A had no adverse effect on the performance of WT mice (FIG. 4F).

The present disclosure demonstrates the lack of adverse effect of CAV-SCN1A in WT mice, suggesting that overexpression of Nav1.1 is well tolerated and possesses strong therapeutic potential on both epileptic and nonepileptic DS phenotypes when administered during the chronic phase.

### Hippocampal Injection of CAV-SCN1A at the Chronic Stage of DS Improves the Symptoms of Epilepsy and Cognitive Abilities.

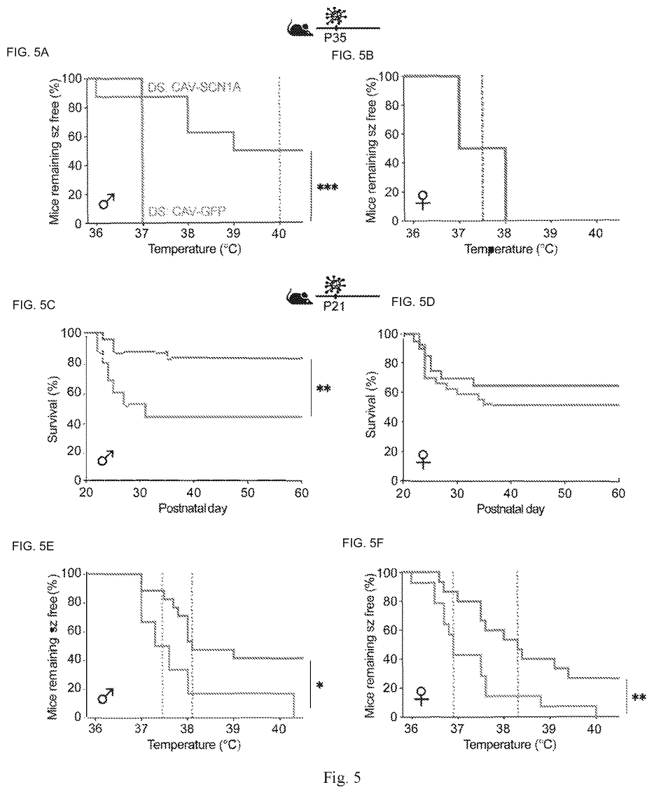

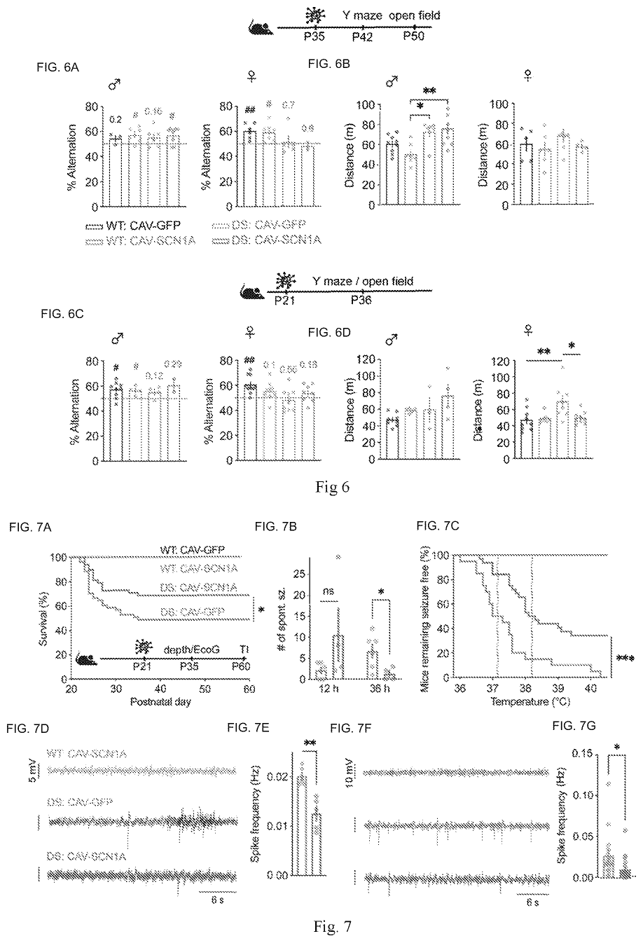

FIG. 4 also illustrates that hippocampal injection of CAV-SCN1A at the chronic stage of DS improves the symptoms of epilepsy and cognitive abilities. As shown in FIG. 4, CAV-GFP or CAV-SCN1A were injected in 5-week-old WT and DS mice. FIGS. 4A-B show data from two weeks after the treatments, when depth electrodes were implanted into the hippocampus at the site of injection. Example traces are shown in FIG. 4A and quantification of the spike frequencies is shown in FIG. 4B. Epileptic activity was not detected in WT mice injected with either CAV-GFP or CAV-SCN1A (n=3 for each); DS: CAV-GFP (n=6); DS: CAV-SCN1A (n=5). FIG. 4 C-D show example traces of cortical ECoG recordings (C) and quantification of the spike frequencies (D). WT: CAV-SCN1A (n=2); DS: CAV-GFP (n=6); DS: CAV-SCN1A (n=5). FIG. 4E shows percent mice remaining free of thermally-induced seizures. The dotted lines represent median seizure temperature. DS: CAV-GFP, n=13; DS: CAV-SCN1A, n=10. See FIG. 5 for separate analysis of males and females. FIG. 4F-Left: shows spontaneous alternation in the Y maze. The dotted line signifies chance level, expected from random alternation. The markings above the bars indicate statistical analysis using one-sample t test relative to 50%. WT: CAV-GFP (n=9); WT: CAV-SCN1A (n=13); DS: CAV-GFP (n=11); DS: CAV-SCN1A (n=10). FIG. 4F-Right: shows the distance moved in the open field. Statistical analysis utilized one-way ANOVA. WT: CAV-GFP (n=14); WT: CAV-SCN1A (n=13); DS: CAV-GFP (n=13); DS: CAV-SCN1A (n=13). See FIG. 6, for separated analysis for males and females. * p<0.05; ** p<0.01; *** p<0.001.

FIGS. 5A-B illustrate the percent of male (A) and female (B) DS mice remaining free of thermally-induced seizures following hippocampal injections of CAV-GFP or CAV-SCN1A at 5 weeks of age. The dotted lines represent median seizure temperature. DS: CAV-GFP, Male n=8, Female n=2; DS: CAV-SCN1A, Male n=8, Female n=6. FIG. 5 C-D illustrate the survival curve of male (C) and female (D) DS mice injected with either CAV-GFP or CAV-SCN1A at P21. DS: CAV-GFP, Male n=25, Female n=27; DS: CAV-SCN1A, Male n=25, Female n=20. FIG. 5 E-F illustrate the percent male (E) and female (F) DS mice remaining free of thermally-induced seizures. The dotted lines represent median seizure temperature. DS: CAV-GFP, Male n=6, Female n=14; DS: CAV-SCN1A, Male n=17, Female n=15.

FIG. 6A depicts the percent spontaneous alternation in the Y maze, of males and females, following gene therapy in adolescent mice. The test was performed 5-10 days post injection (median age P42, range P39-P43). The dotted line signifies chance level, expected from random alternation. The markings above the bars indicate statistical analysis using one-sample t test relative to 50%. WT: CAV-GFP, male n=3; female n=6; WT: CAV-SCN1A, male n=6, female) n=7; DS: CAV-GFP, male n=6, female n=5; DS: CAV-SCN1A, male n=8; female n=2. In FIG. 6B, the open field test was performed 9-17 days post injection (median age P50, range P45-P53). Statistical analysis utilized one-way ANOVA followed by Tukey. WT: CAV-GFP, male n 3=9, female n=5; WT: CAV-SCN1A, male n=7, female n=6; DS: CAV-GFP, male n=6, female n=7; DS: CAV-SCN1A, male n=9, female n=4. FIG. 6C shows percent spontaneous alternation of males and females in the Y maze following gene therapy in juvenile mice (P21). This test was performed 8-14 days post injection (median age P36, range P29-P39). The dotted line signifies chance level expected from random alternation. The markings above the bars indicate statistical analysis using one-sample t test relative to 50%. WT: CAV-GFP, male n=9; female n=10; WT: CAV-SCN1A, male n=4, female n=8; DS: CAV-GFP, male n=4, female n=9; DS: CAV-SCN1A, male n=2, female n=8. FIG. 6D shows the distance moved in an open field following gene therapy in juvenile mice. This test was performed 9-17 days post injection (median age P37, range P30-P41). Statistical analysis utilized one-way ANOVA followed by Tukey. WT: CAV-GFP, male n=8; female n=9; WT: CAV-SCN1A, male n=5, female n=7; DS: CAV-GFP, male n=3, female n=8; DS: CAV-SCN1A, male n=9, female n=4.

### Hippocampal Injection of CAV-SCN1A at the Severe Stage of DS Ameliorates the Epileptic Phenotypes

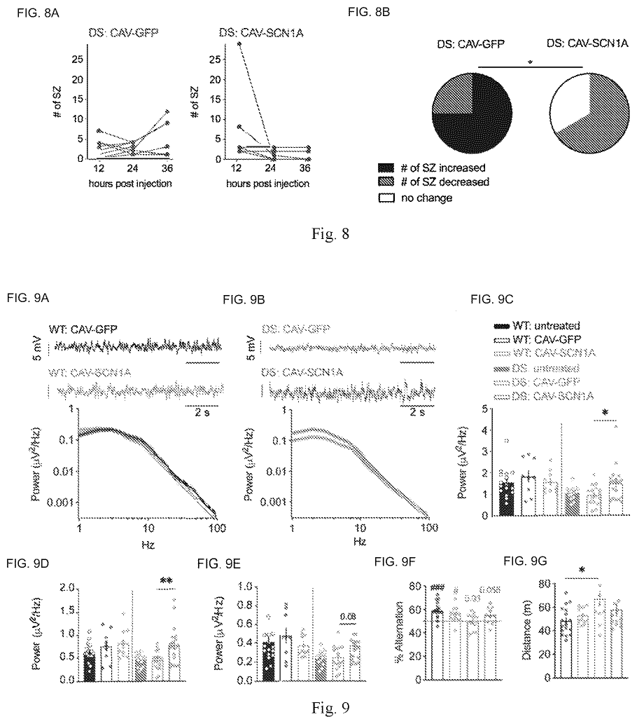

As noted above, the severity of the epileptic phenotypes subsides after the 4^{th} week of life in SCN1A^{A1783V/WT} mice. Thus, it was determined whether exogenous Nav1.1 activity following CAV-SCN1A injections could prevent recurrent spontaneous seizures during the severe stage of the disease, such as in juvenile DS mice younger than 5-weeks old. To that end, bilateral hippocampal injections were performed at the onset of symptoms at P21-P24. Initially, CAV-SCN1A injections reduced the occurrence of premature death by ˜40% compared to mice injected with CAV-GFP (FIG. 7A). Continuous video recordings of a subset of mice, over 24-36 h post-injection, demonstrated that all of the DS mice experienced spontaneous convulsive seizures 12 h post-injection (FIG. 7B, and FIG. 8). However, by 36 h post-injection, DS mice injected with CAV-SCN1A demonstrated a significant (p<0.5) reduction in the number of convulsive seizures, while seizures persisted in those treated with CAV-GFP (FIG. 7B, 36 h and FIG. 8).

Two weeks post-injection, hippocampal and neocortical electrodes were implanted to measure aberrant neuronal activity. It was found that CAV-SCN1A treatment reduced the number of epileptic spikes observed in both regions (FIG. 7 D-G). Treatment with CAV-SCN1A injection in DS mice also reduced the susceptibility to thermally-induced seizures, completely prevented thermally-induced seizures in ˜30% of the treated mice (FIG. 7C), and increased the seizure threshold temperature in mice that retained sensitivity to thermally-induced seizures (FIG. 7C). Together, CAV-mediated expression of Nav1.1, at the severe stage of the DS, improved survival, reduced spontaneous seizures and epileptic spikes occurrence, and increased the temperature threshold of thermally-induced seizures (FIG. 7).

FIG. 7A shows the survival curve of WT and DS littermates injected with either CAV-GFP or CAV-SCN1A at P21-P24. WT: CAV-GFP (n=13); WT: CAV-SCN1A (n=17); DS: CAV-GFP (n=42); DS: CAV-SCN1A (n=48). For separated analysis of males and females, see FIG. 5. FIG. 7B shows the video monitoring of convulsive seizures 12 and 36 h post-injection. DS: CAV-GFP (n=5); DS: CAV-SCN1A (n=4). See FIG. 8 for additional data on individual mice. FIG. 7C shows mice remaining free of thermally-induced seizures. The dotted lines represent median seizure temperature. DS: CAV-GFP (n=20); DS: CAV-SCN1A (n=33). See. FIG. 6 for separated analysis of males and females. FIG. 7 D-G show data from depth electrodes (D, E) or cortical electrodes (F, G) two weeks after treatment. Example traces (FIG. 7 D, F) and quantification of the spike frequencies are also depicted (FIG. 7 E, G). DS: CAV-GFP (n=5 for E, n=16 for G); DS: CAV-SCN1A (n=5 for E, n=19 for G). No epileptic activity was detected in WT mice injected with either CAV-GFP or CAV-SCN1A (n=2 each for depth electrodes and n=9, n=10 respectively, for cortical electrodes. * p<0.05; ** p<0.01; *** p<0.001.

FIG. 8 shows a subset of mice which were recorded on video for 14-36 h post-injection.

FIG. 8A depicts the number of seizures per mouse. The average number of seizures at and 36 h are depicted in FIG. 4B. FIG. 8B shows the change in the frequency of spontaneous seizures over 36 h (Chi-square test p=0.013).

### CAV-SCN1A Injections Rectify Background ECoG Activity and Partially Corrects Working Memory Deficits in DS Mice

Spectral analysis of the ECOG signals 2 weeks post-injection was performed to further explore the impact of exogenous Nav1.1 activity in WT mice, as well as the potential therapeutic effect of this treatment on background, non-epileptic brain oscillations. In WT mice, injection of CAV-GFP or CAV-SCN1A did not alter the spectral ECoG profile, and this power was also similar to that of untreated mice (FIGS. 9A-F; data for untreated mice are replotted from (Fadila et al., 2020)). The present disclosure demonstrates that CAV-2 vectors and exogenous Nav1.1 activity in excitatory and inhibitory neurons do not notably impact global brain oscillations. Mice with DS exhibit an overall lower power of background ECoG activity compared to WT mice (Fadila et al., 2020). Here too, the power of ECOG signals of DS mice injected with CAV-GFP, was lower compared to that of WT mice (p<0.002), and similar to that of untreated DS mice (FIG. 9C, data for untreated mice are replotted from (Fadila et al., 2020)). Conversely, the power of DS mice treated with CAV-SCN1A was higher, mainly in the delta and theta frequency bands (FIG. 9 B-E). Thus, exogenous Nav1.1 activity generated by CAV-SCN1A injections also rectified the background ECoG activity in DS mice.

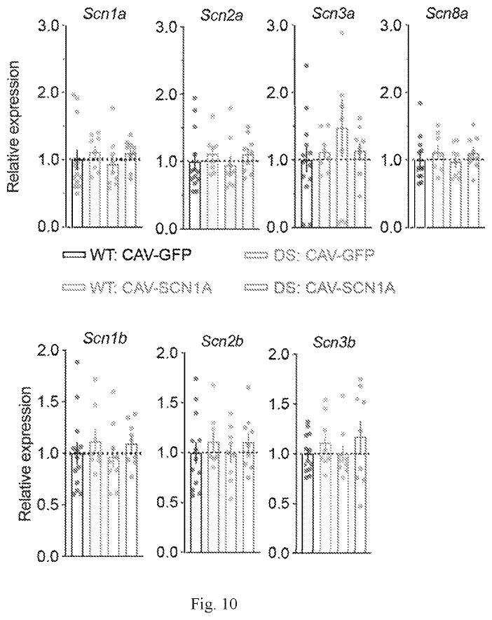

Whether exogenous Nav1.1 activity impacted the expression of other sodium channels was also tested, as it was thought there might be a physiological balance between the levels of sodium channels. It was found that CAV-SCN1A injections did not alter the mRNA level of any other voltage gated sodium channels subtypes, in either control or DS mice (FIG. 10).

Next, the effects of CAV-SCN1A on cognitive abilities were examined. It was found that CAV-SCN1A injections had no adverse effect on the performance of WT mice, with a tendency for improved performance of DS mice in the Y maze (FIG. 9F), as well as a trend for reduced hyperactivity in the open field (FIG. 11G). Together, in addition to the impact on epilepsy, CAV-SCN1A treatment positively affected non-epileptic DS features.

FIGS. 9A-B illustrate examples of background ECoG traces and power density profile of WT (A) and DS (B) mice. FIG. 9 C-E illustrate total power (C, 0.5-100 Hz), the power in the delta (D, 0.5-3.9 Hz) and theta bands (E, 4-8 Hz). Data for untreated mice are replotted from (Fadila et al., 2020). Statistical analysis in panels C-E utilized one way ANOVA (for WT or DS mice separately). WT: CAV-GFP (n=9); WT: CAV-SCN1A (n=10); DS: CAV-GFP (n=16); DS: CAV-SCN1A (n=19). FIG. 9F illustrates spontaneous alternation in the Y maze. The dotted line signifies chance level, expected from random alternation. The markings above the bars indicate statistical analysis using one-sample t test relative to 50%. WT: CAV-GFP (n=19); WT: CAV-SCN1A (n=12); DS: CAV-GFP (n=11); DS: CAV-SCN1A (n=10). FIG. 9G illustrates the distance moved in the open field. Statistical analysis utilized one-way ANOVA: WT: CAV GFP (n=18); WT: CAV2-SCN1A (n=12); DS: CAV-GFP (n=11); DS: CAV-SCN1A (n=13). See FIG. 8, for separated analysis for males and females. * p<0.05; ** p<0.01; *** p<0.001.

FIG. 10 illustrates the relative expression of voltage gated sodium channels. Hippocampi were isolated one month post treatment. For all samples, the expression was first normalized to the expression levels of 2 endogenous controls (Gusb and Tfrc) to calculate ΔCt, followed by normalization to expression in WT mice treated with CAV2-GFP (to calculate ΔΔCt). WT: CAV-GFP n=13; WT: CAV-SCN1A n=8; DS: CAV-GFP n=9; DS: CAV-SCN1A n=9.

Total RNA was isolated using Purelink RNA mini kit according to the manufacturer's instructions (Thermo Fisher Scientific, Life Technologies, Carlsbald, CA, USA). cDNA was synthesized from 500 ng RNA using Maxima H Minus cDNA synthesis kit (Thermo Fisher Scientific, Life Technologies, Carlsbald, CA, USA). For quantitative real-time PCR (qPCR) reactions were performed in triplicates in a final volume of 10 μl with 5 ng of RNA as template using TaqMan gene expression assay (Applied Biosystems, Thermo Fisher Scientific, Life Technologies, Carlsbald, CA, USA) on StepOnePlus® real-time PCR system (Applied Biosystems, Thermo Fisher Scientific, Life Technologies, Carlsbald, CA, USA). The following assays were used: Scn1a (Mm00450580_m1), Scn2a (Mm01270359_m1), Scn3a (Mm00658167_m1), Scn8a (Mm00488110_m1), Scn1b (Mm00441210_m1), Scn2b (Mm01179204_g1), Scn3b (Mm00463369_m1) as well as Gusb (Mm00446953_m1) and Tfrc (Mm00441941_m1), as endogenous controls. Efficiency of 100%, dynamic range, and lack of genomic DNA amplification were verified for all the assays. The relative expression was calculated with 2^{-ΔCT} method using the geomean of endogenous controls, and relative to the expression of WT mice injected with GFP.

### Thalamic Delivery of CAV-SCN1A Revert Epileptic Phenotypes in Juvenile DS Mice

In addition to hippocampal dysfunction, thalamic dysfunction was shown to contribute to network hypersynchrony and DS epilepsy.

Therefore, it was tested whether thalamic injections of CAV-SCN1A also impact DS epilepsy in juvenile mice. Due to the relatively high afference to the thalamus, robust local and widespread transgene expression in WT and DS mice was found (FIG. 11). Again, CAV-SCN1A reduced the occurrence of premature mortality (FIG. 11A) and reduced the susceptibility to thermally-induced seizures (FIG. 11B). Furthermore, cortical ECoG recordings demonstrated that exogenous Nav1.1 activity reduced the frequency of epileptic spikes (FIG. 11 C, D) and corrected the power of background activity (FIG. 11E). The present disclosure demonstrates improvement of epileptic phenotypes and global brain oscillations following CAV SCN1A injection into the thalamus or the hippocampus of DS mice.

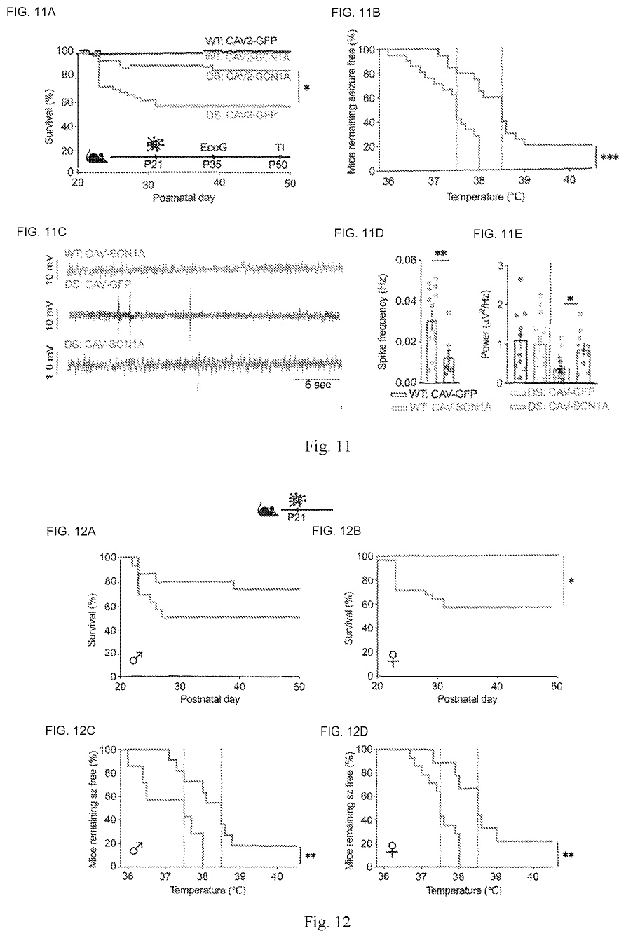

FIG. 11A illustrates the survival curve of WT and DS littermates injected with either CAV-GFP or CAV-SCN1A. WT: CAV-GFP (n=24); WT: CAV-SCN1A (n=31); DS: CAV-GFP (n 8=44); DS: CAV-SCN1A (n=25). FIG. 11B illustrates the percentage of mice remaining free of thermally-induced seizures. The dotted lines represent median seizure temperature. DS: CAV-GFP (n=28); DS: CAV-SCN1A (n=20). See FIG. 12 for separated analysis of males and females. FIG. 11 C-D illustrate data from two weeks after the treatments when cortical electrodes were implanted. Example traces (FIG. 11C) and quantification of the spike frequencies are depicted (FIG. 11D). FIG. 11E illustrates the total ECoG power (0.5-100 Hz) WT: CAV-GFP (n=11); WT: CAV-SCN1A (n=13); DS: CAV-GFP (n=14); DS: CAV-SCN1A (n=10).

FIGS. 12A-B show percent survival curve of male (A) and female (B) DS mice injected with either CAV-GFP or CAV-SCN1A at P21. DS: CAV-GFP, male n=16, female n=28; DS: CAV-SCN1A, male n=15, female n=10. FIG. 12 C-D shows percent male (C) and female (D) DS mice remaining free of thermally-induced seizures. The dotted lines represent median seizure temperature. DS: CAV-GFP, male n=7, female n=14; DS: CAV-SCN1A, male n=11, female n=9.

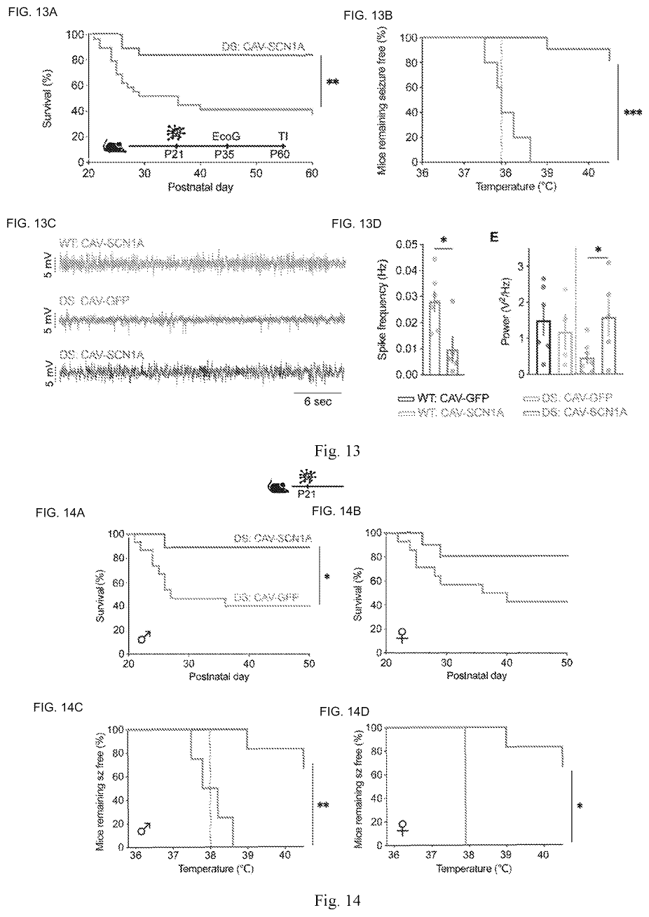

Next, it was determined whether CAV-SCN1A injections into the thalamus and hippocampus could further enhance the therapeutic effect of exogenous Nav1.1 activity. Indeed, following these dual deposits along the needle track, DS mice treated with CAV-SCN1A demonstrated over 80% changes of survival (FIG. 13A) as well as notable protection from thermally-induced seizures, with only two mice that had thermal seizures out of 11 mice that were tested (FIG. 13B). Moreover, similar to the effect following hippocampal or thalamic administration, CAV-SCN1A reduced the number of epileptic spikes and increased the power of background ECoG.

Combined Thalamic and Hippocampal Injection of CAV-SCN1A in Juvenile Mice Protects from Thermally-Induced Seizures

Together, treatment of juvenile DS mice with CAV-SCN1A injections into hippocampus or the thalamus dramatically ameliorates their epileptic phenotypes. However, combined delivery provided greater protection from SUDEP and thermally-induced seizures.

FIG. 13 demonstrates that the combined thalamic and hippocampal injection of CAV-SCN1A into juvenile mice protects from thermally-induced seizures. FIG. 13A shows the survival of DS mice injected with either CAV-GFP or CAV-SCN1A. DS: CAV-GFP (n=28); DS: CAV-SCN1A (n=19). FIG. 13B shows the percentage of Mice remaining free of thermally-induced seizures. The dotted lines represent median seizure temperature. DS: CAV-GFP (n=5); DS: CAV-SCN1A (n=11). See FIG. 14 for separated analysis of males and females. FIG. 13 C-D illustrate data from two weeks after the treatments when cortical electrodes were implanted. Example traces (FIG. 13C) and quantification of the spike frequencies are depicted (FIG. 13D). FIG. 13E illustrates total ECOG power (0.5-100 Hz) WT: CAV-GFP (n=5); WT: CAV-SCN1A (n=4); DS: CAV-GFP (n=6); DS: CAV-SCN1A (n=10).

FIGS. 14A-B show the survival curve of male (A) and female (B) DS mice injected with either CAV-GFP or CAV-SCN1A at P21. DS: CAV-GFP, male n=15, female n=14; DS: CAV-SCN1A, male n=9, female n=10. FIG. 14 C-D show the percent of male (C) and female (D) DS mice remaining free of thermally-induced seizures. The dotted lines represent median seizure temperature. DS: CAV-GFP, male n=4, female n=1; DS: CAV-SCN1A, male n=5, female n=6.

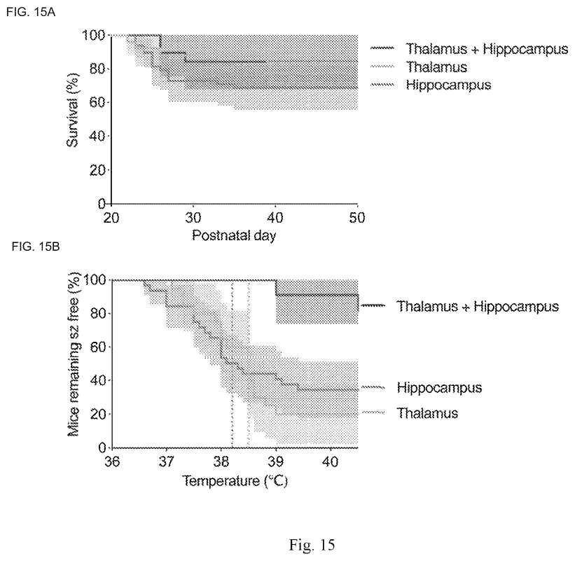

FIG. 15A illustrates that there were no statistical differences in the survival of DS mice following CAV-SCN1A injections into the hippocampus, the thalamus, or dual injections into both locations. The solid lines are the same data presented in FIGS. 7A, 11A, 13A, respectively, and the shaded areas depicts 95% confidence intervals. FIG. 15B illustrates that the injection of CAV-SCN1A into both the thalamus and hippocampus provides greater protection from thermally-induced seizures. The solid lines are the same data presented in FIGS. 7C, 11B, 13B, respectively, and the shaded areas depicts 95% confidence intervals.

### Hippocampal Injection of CAV-NSE-SCN1A in Alzheimer's Mouse Models Reverts Mouse Behavior and Histopathological Markers to Near Wildtype Levels

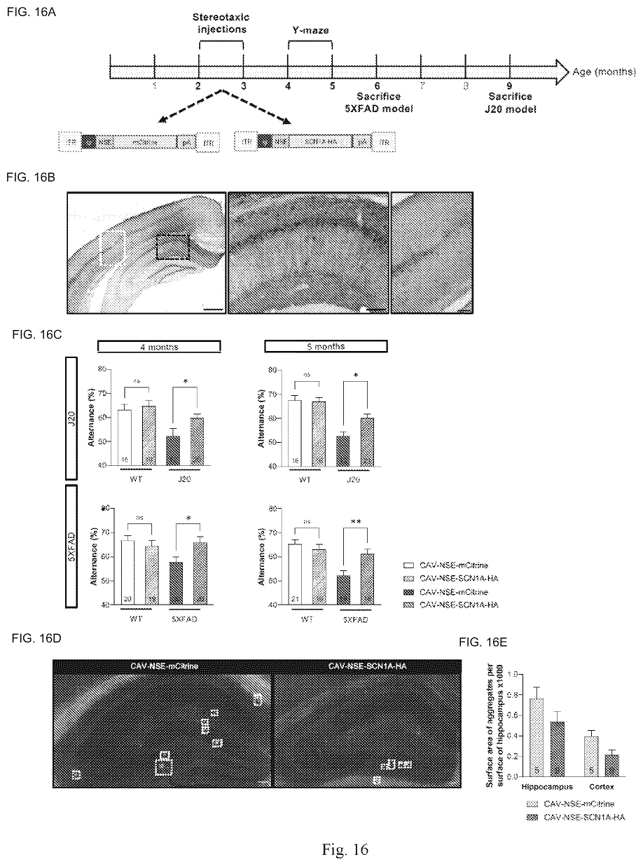

FIG. 16A illustrates a design of a study in which two CAV-2 vectors, harbouring an mCitrine cassette or a codon-modified SCN1A ORF with fused HA tag, are bilaterally injected in the hippocampus of Alzheimer's mouse models (5XFAD and J20). 5XFAD mice carry a human amyloid precursor protein (APP) transgene and presenilin 1 transgenes, and develop plaques beginning at 2 months. J20 mice express the human APP695/751/770 containing the Swedish double mutations (K670N/M671L) at the β-secretase cleavage site and the V717F mutation at the γ-secretase cleavage site. The transgene is driven by a PDGFβ promoter. J20 mice develop plaque deposition and cognitive deficit at ˜6 months. ITR: inverted terminal repeat. ψ: packaging signal. NSE (promoter): neuron-specific enolase. pA: polyadenylation signal.

In FIG. 16B, the vectors (˜1 microlitre containing 10⁹ vector particles) are injected into the hippocampus using stereotactic apparatus. Exogenous Nav1.1 is detected using immunohistology against the HA tag. Second and third columns are a higher magnification of, respectively, black & white frames of the first column.

FIG. 16C demonstrates the physiological effect of CAV-2 mediated exogenous Nav1.1 on working memory in the Y-maze test in 4 and 5-months-old J20 and 5XFAD mice. The Y Maze is a behavioural test measuring the willingness of rodents to explore new environments. Testing is in a Y-shaped maze with three arms at a 120° angle from each other. A mouse is allowed to freely explore the arms. Over the course of multiple entries, the mouse should show a tendency to enter a less recently visited arm. The Y Maze allows one to quantify cognitive deficits and evaluate novel entities for their effects on cognition. Many parts of the brain, in particular the hippocampus, are involved in this task.

FIG. 16D-E depict and demonstrate amyloid loads (white frames) as identified by Thioflavin S analyses, and quantification in the hippocampus and cortex of 9-month-old J20 mice injected with the two vectors.

### Discussion

DS is an intractable childhood epileptic encephalopathy, with a high mortality rate compared with other developmental epilepsies. SUDEP is the leading cause of death, with most occurrences before the age of 10. Importantly, pharmacological seizure control in DS is notoriously difficult, despite polytherapy and recent advancements in therapeutic options. Therefore, there is an urgent need for novel treatments. In addition to the life-threatening epileptic phenotypes, individuals with DS suffer from non-epileptic comorbidities, including developmental delays, cognitive impairment, and hyperactivity. While these behavioral deficits greatly impact the quality of life of patients and families, the therapeutic toolbox for addressing these burdening issues is limited.

According to the present disclosure, CAV-mediated expression of Nav1.1, via a codon-modified SCN1A ORF significantly improved comorbidities in juvenile and adolescent DS mice, ameliorated the epileptic phenotypes, corrected background ECoG activity, and improved cognitive functions. Notably, the present disclosure relates to the only treatment method that is effective during the severe and chronic stages of DS.

C57BL/6J-Scn1a^{A1783V/WT} mice display severe epileptic phenotypes compared to other DS mouse models (Han et al., 2020; Niibori et al., 2020), which may render this model particularly resistant to treatment. Specifically, similarly to patients with DS, all the mice experience spontaneous seizures (FIG. 10) and thermally-induced seizures within the range of clinical febrile seizures. Furthermore, while the mortality of these mice is higher compared to the risk reported in patients (FIGS. 7, 11, 13), it should be noted that the mice do not receive any anti-seizure medications or emergency care with prolonged seizures. Thus, the present disclosure relates to a treatment method for severe epileptic phenotypes which faithfully represent the clinical severity of DS in humans and which treatment can be combinable with traditional anti-seizure medications to potentially achieve even greater therapeutic results. Moreover, in contrast to other models that harbor SCN1A truncation mutations, effective gene therapy treatment in this model should overcome the persistent membrane expression of the malfunctioning mutant SCN1A allele. The present disclosure demonstrates the therapeutic impact of CAV-SCN1A despite the challenges of this DS model and highlights the clinical potential of this treatment method and vector.

Notably, in contrast to previous efforts to provide gene-specific treatments for DS, which merely tested therapeutic effect when administered during the asymptomatic, pre-epileptic stage, or during the chronic phase, CAV-SCN1A has a demonstrated effect at multiple disease stages (FIGS. 4-13). At the severe stage of the disease, CAV-SCN1A improved survival, reduced the occurrence of spontaneous seizures, reduced the frequency of epileptic spikes and protected against thermally-induced seizures (FIGS. 7, 11, 13). At the chronic stage of the disease, as the frequency of spontaneous seizures and occurrence of SUDEP is low, amelioration of the epilepsy was demonstrated as reduced frequency of epileptic spikes and reduced sensitivity to thermally-induced seizures (FIG. 4). Moreover, as this treatment relies on vector-mediated SCN1A ORF delivery, rather than transcriptional activation of the one functional Nav1.1 copy, it should be suitable for patients with both truncation and missense mutations in SCN1A.

Complete cure of DS epilepsy is extremely challenging. Antisense oligonucleotide treatment to enhance SCN1A transcription, administered at P2, well within the asymptomatic period, prevented premature mortality but spontaneous seizures were still observed. Moreover, when said treatment was given at P14, still in the midst of the pre-epileptic stage, full protection from death was also not achieved. Similarly, epileptic spikes and spontaneous seizures were still detected following neonate dCas9-based SCN1A activation, and premature death (despite improved survival) was still evident following viral mediated overexpression of voltage-gated sodium channels beta subunits. Likewise, abnormal epileptic activity still persisted when the treatments were administered during the chronic stage of the disease (FIG. 4).

Notably, CAV-SCN1A injections in the hippocampus or the thalamus, had similar effect on epilepsy (FIGS. 7, 11), implicating these areas in DS pathophysiology. Combined delivery into these two regions had similar protection from premature mortality (FIG. 15A), but greater protection from thermally-induced seizures (FIG. 15B). This is possibly due to larger biodistribution of the exogenous functional Nav1.1. Interestingly, distinct circuit-specific neuronal dysfunctions were described for each of these regions. Disinhibition was indicated as the culprit in the hippocampus. Conversely, complex neuronal changes were reported in the thalamus, with reduced activity of inhibitory and excitatory neurons, as well as hyperexcitability of inhibitory thalamic reticular nucleus neurons that led to augmented cortico-thalamic oscillations and seizures. Thus, despite the seemingly opposing local neuronal dysfunction, an unexpected and superior therapeutic effect of CAV-SCN1A was observed, which further highlights the efficacy and potential of this treatment.

One significant challenge for CNS-targeted gene delivery is the need to transduce enough neurons within a critical brain region, to trigger a global change in network function. The present disclosure further demonstrates the pivotal role of Nav1.1 expression within the injected sites and the widespread therapeutic effect of transfection with CAV vectors, and their retrograde expression capabilities, leading to widespread CNS gene expression. Despite only two injection sites, with CAV's unique properties, therapeutic effect is seen even with only a relatively small number of neurons transduced.

In one aspect, the present disclosure demonstrates the positive global effect triggered by restoring Nav1.1 expression within a tightly connected neuronal network that hubs at an injection site but includes multiple projecting neurons, mostly excitatory, from various additional brain regions (FIGS. 1, 2). This corrected Nav1.1 expressing neuronal ensemble can now prevent the generalization of epileptic activity, with additional corrective contribution to neuronal networks that are not directly transduced, accounting for the ability of this treatment to revert the epilepsy, correct background neuronal activity, and positively influence cognitive abilities. Furthermore, it can also account for the wide time-window for therapeutic intervention, and the ability to revert the symptoms even in adolescent mice. In some aspects, the present disclosure further illustrates that disruption of the activity of single neurons within a connected hub can disrupt epileptic network dynamics, indicating that transducing all the neurons may not be needed for effective therapy.

According to certain embodiments, the present disclosure relates to CAV-mediated SCN1A delivery as a therapeutic approach for children and adolescent individuals with DS-associated SCN1A missense and truncation mutations. The disclosure relates to a vector for the reversion of DS or conditions thereof. In certain embodiments, the vector comprises a first nucleic acid sequence encoding parts of a CAV-2 genome and a second nucleic acid sequence encoding a functional Nav1.1 sodium channel.

In certain embodiments, the viral vector can also comprise a first regulatory sequence encoding a transcriptional control region. The transcriptional control region can optionally include a promoter and/or enhancer region for transcriptional control. In certain embodiments, the promoter region comprises a promoter sequence encoding at least one promoter selected from the group consisting of CAG, hSyn, NSE, and Dlx5/6. In certain embodiments, the second nucleic acid sequence is operably linked to the first regulatory sequence for expression of the functional Nav1.1 sodium channel in mammalian cells. In certain embodiments, the first regulatory sequence is controllable to drive positive expression of the Nav1.1 sodium channel. The present disclosure relates to a viral vector that is configured for transcriptional regulation and which can drive positive regulation and/or negative regulation of expression of exogenous functional Nav1.1 sodium channel.

According to certain embodiments, the second nucleic acid sequence is an engineered SCN1A ORF created by eliminating at least one short repeat sequence from native SCN1A. In some embodiments, the second nucleic acid sequence is created by eliminating at least one short repeat sequence encoding a transmembrane section of the functional Nav1.1 sodium channel. This disclosure relates to an engineered CAV-2 SCN1A vector wherein regions of the vector have been engineered, such as through deletion of short repeat sequences, to prevent SCN1A rearrangement when subcloned into a plasmid and propagated.

The disclosure also relates to a method of promoting reversion of DS diseases or conditions thereof, comprising administering to a mammal a therapeutically effective amount of the CAV-2 SCN1A viral vector. The method results in a vector-induced change in the balance of neural network activity. The mammal preferably includes humans as well as other species suitable for treatment and/or scientific research such as mice, rats, pigs, monkeys, or other suitable mammals. In certain embodiments, the viral vector is administered into a central nervous system of the mammal. It is also envisioned that the viral vector can be administered into at least one brain region. In some embodiments, the at least one brain region can be selected from the group consisting of hippocampus and thalamus. However, the disclosure is not intended to be limited, and it is envisioned that the vector can be effectively administered to other regions of the brain or CNS as well to promote positive clinical outcomes. It is a feature of the disclosure that the viral vector can be administered prior to development of a chronic form of DS disease or conditions thereof, thereby preventing premature death and promoting reversion of DS and symptoms thereof.

The present disclosure relates to a CAV-2 SCN1A vector which can be administered to children or adolescent humans with a DS-associated missense or truncation mutation in a SCN1A gene. In certain embodiments, after administration of the viral vector, the mammals are monitored for seizure reduction, and if desired clinical results are not obtained, a second therapeutically effective amount of the vector can be administered. Desirable clinical results are seen, for example, by way of corrected expression of Nav1.1, DS reversion, seizure reduction, as well as improvements in the non-epileptic phenotypes of DS including cognitive deficit and hyperactivity. In one aspect of this disclosure, the method can include the co-administration of a therapeutically effective amount of anti-seizure medication.

The disclosure also relates to a composition for corrected expression of Nav1.1 and conditions thereof, such as, by way of example and so as not to limit the scope, DS, epilepsy, or Alzheimer's. In certain embodiments, the composition includes a viral vector for the treatment of pathological conditions of SCN1A mutation and Nav1.1 sodium channel miscoding, the viral vector comprising a first nucleic acid sequence encoding a CAV-2 vector and a second nucleic acid sequence encoding a functional Nav1.1 sodium channel. In certain embodiments, the viral vector optionally includes a first regulatory sequence, the second nucleic acid sequence operably linked to the first regulatory sequence for controlled expression of the Nav1.1 sodium channel in mammalian cells. In certain embodiments, the first regulatory sequence optionally includes a promoter region comprising a promoter sequence encoding one of the aforementioned promoters, such as NSE. It is envisioned that other promoters and/or enhancers may be used, for example, promoters and/or enhancers which are suitable for preferential expression of SCN1A within neurons.

In certain embodiments, the disclosure relates to a vector for the compensation of loss of function of endogenous Nav1.1 and treatment of pathological conditions associated with SCN1A mutation and Nav1.1 sodium channel miscoding. In some embodiments, the vector comprises a first nucleic acid sequence encoding at least one portion of a CAV-2 genome; a second nucleic acid sequence encoding a functional Nav1.1 sodium channel; a first regulatory sequence encoding a transcriptional control region, the transcriptional control region comprising a promoter or enhancer or elongation factor sequence encoding at least one promoter/enhancer/elongation factor selected from the group consisting of E2, E6, EF1, CAG, hSyn, Dlx5/6, and NSE, and preferentially, NSE; wherein the second nucleic acid sequence is operably linked to the first regulatory sequence for controlled transcriptional expression of an SCN1A open reading frame (ORF) in mammalian cells.

In certain embodiments, the vector can be designed such that the second nucleic acid sequence is an engineered SCN1A ORF; and wherein the engineered SCN1A ORF is created by eliminating at least one short repeat sequence from a codon-modified SCN1A ORF. In some embodiments, the second nucleic acid sequence is a sequence having at least 50%, 55%, 60%, 65%, 70%, 75%, 80%, 85%, 90%, or 95% homology with nucleic acid sequences selected from group consisting of Sequence ID Number 12 and Sequence ID Number 14. In other embodiments, the second nucleic acid sequence is a sequence encoding for a protein having at least 50%, 55%, 60%, 65%, 70%, 75%, 80%, 85%, 90%, or 95% homology with amino acid sequences selected from group consisting of Sequence ID Number 15 and Sequence ID Number 16.

In other embodiments, the vector can be designed such that the second nucleic acid sequence is created by eliminating at least one short repeat sequence in an ORF encoding a transmembrane section of the functional Nav1.1 sodium channel.

In more embodiments, the vector is therapeutically active in children or adolescent individuals with misexpression of the gene encoding Nav1.1, such as in the case of Dravet Syndrome-associated SCN1A missense or truncation mutations, as well as in other pathological conditions such as epilepsy or Alzheimer's.

In certain embodiments, the vector can be optimized for preferential expression of SCN1A ORF in neurons and/or configured for transcriptional regulation. In certain embodiments the SCN1A ORF encodes amino acid SEQ ID NOS 15 and 16, which encode WT and Synthetic Nav1.1 proteins, respectively, lacking the short amino acid sequence VIIDKPATDDN. These amino acid sequences are typically expressed in the brain, whereas, for example, SEQ ID NOS 12 and 14 encode genetic sequences for the expression of somatic Nav1.1.

The disclosure further relates to a method of compensating for the loss of function of Nav1.1 and treatment of pathological conditions associated with SCN1A mutation and Nav1.1 sodium channel miscoding, including administering a therapeutically effective amount of a vector to a mammal. In some embodiments, the vector can be administered into a central nervous system of the mammal, or into at least one brain region. In certain embodiments, the vector can be administered into at least one brain region selected from the group consisting of hippocampus and thalamus. In other embodiments, the vector can be administered prior to development of a chronic form of Dravet Syndrome disease or comorbidities thereof. In certain embodiments, the vector can be administered to treat a chronic form of Dravet Syndrome disease or comorbidities thereof.

In certain embodiments, after administration of the vector, the method further includes inducing expression of the functional Nav1.1 sodium channel. In other embodiments, the vector can be administered to children, adolescent, adult, or elderly mammals with a missense or truncation mutation in an SCN1A gene. In certain embodiments, after administration of the vector, the method further includes monitoring the mammal for desired clinical results, such as amelioration or improvement in pathological conditions—such as DS, epilepsy, Alzheimer's and other Nav1.1 associated pathological disorders—and if the desired clinical results are not obtained, administering a second therapeutically effective amount of the vector until therapeutic results are obtained.

In some embodiments, the vector is deposited at the injection site, and the vector can be delivered to the injection site and multiple sites that contain projecting neurons from various brain regions. In some embodiments, the treatment method further includes administering a therapeutically effective amount of anti-seizure, epilepsy, or Alzheimer medication.

In some embodiments, the therapeutically effective amount of the vector can be administered for the treatment of a pathological condition selected from the group consisting of Dravet Syndrome, Epilepsy, and Alzheimer's. Additionally, this method could be beneficial in cases where modifying the brain neuronal network reduces disease symptoms (e.g., Autism Spectrum Disorder (ASD).

The disclosure further relates to a composition the compensation of loss of function of Nav1.1 and the treatment of SCN1A mutation and Nav1.1 sodium channel miscoding, the composition including a viral vector, the vector comprising a first nucleic acid sequence encoding at least one portion of a CAV-2 genome and a second nucleic acid sequence encoding a functional Nav1.1 sodium channel; wherein the vector optionally includes a first regulatory sequence, the second nucleic acid sequence operably linked to the first regulatory sequence for controlled transcriptional expression of an SCN1A ORF in mammalian cells; and wherein the first regulatory sequence includes a transcriptional control region comprising a promoter/enhancer/elongation factor sequence encoding at least one promoter/enhancer/elongation factor selected from the group consisting of E2, E6, EF1, CAG, hSyn, Dlx5/6, and NSE, and preferably, NSE.

### Methods

### Vectors Generation

All CAV-2 vectors used in this work were generated using a seamless ligation cloning extract (SLICE) strategy (Zhang et al., 2012). The following promoters were obtained from Addgene: the NSE promoter (plasmid #50958 James Bamburg, (Mi et al., 2013)); the CAG promoter (plasmid #51274, Pawel Pelczar, (Hermann et al., 2014)); hSyn promoter (plasmid #22909, Edward Callaway, (Nathanson et al., 2009)); the Dlx5/6 enhancer (plasmid #83900, Gordon Fishell, (Dimidschstein et al., 2016)) Citrine, SCN1A cDNA (Mora-Jimenez et al., 2021) and the bovine growth hormone polyA sequence. Fragments were subcloned into the E1 region of E1/E3-deleted pCAV-2. Then, vectors were expanded and purified as previously described (Ibanes and Kremer, 2013; Kremer et al., 2000).

### Animals

All animal experiments were approved by the institutional care and use committee of Tel Aviv University and CNRS. WT and DS mice harboring the global Scn1aA1783V mutation were generated by crossing conditional Scn1aA1783V males (The Jackson Laboratory; stock #0216133) with CMV-Cre females (stock #006054). Mice were housed in a standard animal facility at the Glodschleger Eye institute at a constant temperature of 22° C., on a 12-hour light/dark cycle, with ad libitium access to food and water.

### IH

It is worthwhile repeating that CAV-2-mediated gene transfer is linked to CAR expression pattern on neurons (Soudais et al., 2000; Zussy et al., 2016). As the levels of CAR changes during development and in inflammatory diseases like Alzheimer's, it was possible that CAV-2 vector efficacy differs in adolescent and juvenile healthy or DS mice. Therefore CAV-GFP was injected into juvenile healthy and DS mice and transgene expression was examined. In DS mice, similar robust transgene expression at the site of injection and in afferent regions (hippocampus, neocortex, striatum, internal capsule, and thalamus) was found.

### Viral Injections

WT and DS mice at the age of P21-24, or P35-P36 were randomly assigned to treatment with CAV-2-NSE-GFP (CAV-GFP) or CAV-2-NSE-SCN1A (CAV-2-SCN1A). The mice were anesthetized using ketamine/xylazine (191/4.25 mg/kg), Carprofen (5 mg/kg) was used for analgesia. The mice were placed in a stereotaxic device (Ultra Precise Stereotaxic Instruments, Stoelting, Wood Dale, IL, USA). A midline incision was made above the skull and holes were made using a 25 G needle in the place of injection. For hippocampal injections we used the following coordinates: anterior/posterior (AP): −1.8; medial/lateral (ML): ±1.7; dorsal/ventral (DV): −3 for P21-24 and AP: −1.7; ML: ±1.8; DV: −3 for P35-36. For thalamic injections at P21-P24 we use the following coordinates: AP: −1.4; ML: ±2.2 DV: −3.5. 1 ul containing 1×10\hat{ }9 vp was injected on each side, using a 1 ul beveled needle Hamilton syringe (1 ul syringe, 7000 series, beveled tip; For dual thalamic-hippocampal injections the injection needle was lowered to: ML: ±1.8; PL: ±1.8; DV: −3.5, injected with 0.5 ul, following a few min, the injection needle was raised to DV: 3, and injected with 0.5 ul. The AP coordinates were slightly modified for P21 mice in which the distance between bregma and lambda was less than 3 mm, and reduced to AP: −1.7.

The DV coordination was measured from the tip of the beveled injection needle) at a rate of 100 nL/min (Quintessential Stereotaxic Injector, Stoelting, Wood Dale, IL, USA). After injection, the syringe was kept in place for at least 5 minutes to prevent backflow before it was slowly retracted. The skin was then closed with sutures and the mice were isolated for a period of 7 days according to TAU BSL-2 safety instructions.

### ECoG and Depth Electrode and Recordings

One week following viral injection, cortical or depth electrodes were implanted as previously described (Fadila et al., 2020). Briefly, a midline incision was made above the skull, and fine silver wire electrodes (130 mm diameter bare; 180 mm diameter coated) were implanted. We used the previously formed injection holes for ECOG or depth electrodes for hippocampal depth recordings, the wire electrodes were lowered using the same stereotactic coordinates used for injection. A reference electrode was placed on the cerebellum; and a ground electrode was placed subsequently behind the neck. The electrodes were connected to milmax-connector, secured with dental cement, the skin was closed with sutures. The mice were given at least wo days to recover before recording.

Video-depth/EcoG recordings lasted 2-4 hours, during the light period, were obtained from freely moving mice, connected to a T8 Headstage (Triangle BioSystems, Durham, NC, USA), using a PowerLab 8/35 acquisition hardware and the LabChart 8 software (ADInstruments, Sydney, Australia). The electrical signals were recorded and digitized at a sampling rate of 1 KHz with a notch filter at 50 Hz. The analysis was performed using LAbChart 8 (ADInstrumnts, Sydney, Australia). The ECOG signal was processed offline with a 0.5-100 Hz bandpass filter. Power spectral density was calculated using fast Fourier transform, with Hann (cosine-bell) data window set to 50% overlap. For each mouse, five to eight 30 s long segments of the wakefulness, immobile and epileptic-free, but following a movement as determined by the video recording, were averaged.

### Thermally-Induced Seizures

Thermal induction was done as described before (Almog et al., 2021) one-month post-injection. Briefly, the mice were given 10 minutes to habituate to the thermal probe and the recording chamber. The baseline body temperature was measured, followed by an increase of 0.5° C. every 2 minutes until 40.5° C. or until a seizure was generated.

### Behavioral Experiments

Behavioral experiments were performed as described before (Fadila et al., 2020). Spontaneous alternation in the Y maze was performed 5-14 days post-injection and the open field test was performed 9-17 days. Briefly, for the Y-Maze spontaneous alternation test the mice were placed in a symmetrical Y-maze comprised of three opaque white Plexiglas arms (each 35 cm L×7.6 cm W×20 cm H) and allowed free exploration for 10 min. For the Open-field test, mice were placed in the center of a square (50×50 cm) Plexiglas apparatus and their activity over was recorded for 10 min. Live tracking was achieved via a monochrome camera (Basler acA1300-60 gm, Basler AG, Ahren, Germany) connected with Etho Vision XT 13 software (Noldus Technology, Wageningen, Netherlands).

### SEQUENCE LISTING

| Sequence ID Number | Sequence Name | Sequence Type |
|---|---|---|
| 1 | BGHpA (Bovine Growth | DNA |
| | Hormone polyAdenylation) | |
| 2 | CAG Promoter | DNA |
| 3 | CAV2 | DNA |
| 4 | CMV Promoter | DNA |
| 5 | E2 Enhancer | DNA |
| 6 | E6 Enhancer | DNA |
| 7 | EF1a Promoter | DNA |
| 8 | hSyn Promoter | DNA |
| 9 | mDlx Enhancer | DNA |
| 10 | NSEr Promoter | DNA |
| 11 | WT SCN1A Somatic | AA |
| 12 | WT SCN1A | DNA |
| 13 | xSCN1A from GeneScript ™ | AA |
| | Somatic | |
| 14 | xSCN1A from GeneScript | DNA |
| 15 | WT SCN1A Brain | AA |
| 16 | xSCN1A from GeneScript ™ | AA |
| | Brain | |

### Definitions

As used herein, the singular forms “a”, “an”, and “the” include plural references unless indicated otherwise. For example, “a” component in a composition includes one or more components.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase. A polynucleotide may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, ply-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), “(O)NR 2 (“amidate”), P(O)R, P(O)OR′, CO or CH 2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

“Treatment” can be assessed using any endpoint indicating a benefit to the patient, including, without limitation, (1) inhibition, to some extent, of disease progression, including slowing down and complete arrest; (2) reduction in the number of disease episodes and/or symptoms; (3) reduction in comorbidity episodes and/or symptoms; (4) inhibition (i.e., reduction, slowing down or complete stopping) of disease and/or comorbidity symptom progression; (5) inhibition (i.e., reduction, slowing down or complete stopping) of comorbidity progression; (6) relief, to some extent, of one or more symptoms associated with a disorder or with comorbidities thereof; (7) increase in the length of disease-free presentation following treatment; (8) decreased mortality at a given point of time following treatment; and/or (9) lack of adverse effects following treatment. Treatment can also be assessed using any endpoint indicating side effect and/or toxicity to the patient.

“Treating” or “treatment” or “alleviation” refers to therapeutic treatment wherein the object is to slow down (lessen) if not cure the targeted pathologic condition or disorder or prevent recurrence of the condition. A subject is successfully “treated” if, after receiving a therapeutic amount of a therapeutic agent, the subject shows observable and/or measurable reduction in or absence of one or more signs and symptoms of the particular disease or comorbidities thereof. Reduction of the signs or symptoms of a disease or comorbidities may also be felt by the patient. Treatment can achieve a complete response, defined as disappearance of all signs a pathological condition, or a partial response, wherein the symptoms or presence of a pathological condition is reduced, preferably by more than 50 percent, more preferably by 75%. A patient is also considered treated if the patient experiences stable disease. In some embodiments, treatment with a therapeutic agent is effective to result in the patients being disease-free 3 months after treatment, preferably 6 months, more preferably one year, even more preferably 2 or more years post treatment. These parameters for assessing successful treatment and improvement in the disease are readily measurable by routine procedures familiar to a physician of appropriate skill in the art.

It is understood that aspects and embodiments of the invention described herein include “comprising”, “consisting”, and/or “consisting essentially of” aspects and embodiments.

1. A vector for providing exogenous Nav1.1 sodium channels, comprising:
a first nucleic acid sequence encoding at least one portion of a CAV-2 genome;
a second nucleic acid sequence encoding a functional Nav1.1 sodium channel;
a first regulatory sequence encoding a transcriptional control region, the transcriptional control region comprising a promoter/enhancer/elongation factor sequence encoding at least one promoter/enhancer/elongation factor selected from the group consisting of an endogenous SCNIA promoter, an endogenous SCNIA enhancer, an endogenous SCNIA transcription factor, E2, E6, EF1, CAG, hsyn, Dlx5/6, and NSE, and preferably, NSE;
wherein the second nucleic acid sequence is operably linked to the first regulatory sequence for controlled transcriptional ex an SCN1A open reading frame (ORF) in mammalian cells.

2. The vector of claim 1, wherein the second nucleic acid sequence is Sequence ID Number 12 or Sequence ID Number 14, or encodes for Sequence ID Number 15 or Sequence ID Number 16.

3-5. (canceled)

6. The vector of claim 1, wherein:
the second nucleic acid sequence is an engineered SCNIA ORF;
and wherein the engineered SCNIA ORF is created by eliminating at least one short repeat sequence from a codon-modified SCNIA ORF, or
the second nucleic acid sequence is created by eliminating at least one short repeat sequence in an ORF encoding a transmembrane section of the functional Nav1.1 sodium channel.

7. (canceled)

8. The vector of claim 1, wherein the vector is designed for use in the treatment of pathological conditions associated with SCNIA mutation or network dysfunction.

9. (canceled)

10. The vector of claim 1, wherein the vector is therapeutically active in individuals with SCNIA missense mutations or SCNIA truncation mutations, or with neuronal dysfunction, or with a loss of function of endogenous Nav1.1 activity, or with a pathological condition selected from the group consisting of Dravet Syndrome, epilepsy, Alzheimer's, and autism spectrum disorder.

11. The vector of claim 1, wherein the vector is optimized for preferential expression of SCN1A ORF in neurons.

12-17. (canceled)

18. A method for providing exogenous Nav1.1 channels, comprising:
administering a therapeutically effective amount of the vector of claim 1 to a mammal, possibly into a central nervous system of the mammal or into at least one brain region, possibly selected from the group consisting of hippocampus and thalamus.

19-21. (canceled)

22. The method of claim 18, wherein the vector is administered prior to development of Dravet Syndrome phenotypes or comorbidities thereof, or after development of Dravet Syndrome phenotypes or comorbidities thereof.

23. (canceled)

24. The method of claim 18, wherein the vector is administered to treat Dravet Syndrome or comorbidities thereof, or to prevent the development of Dravet Syndrome or comorbidities thereof.

25. (canceled)

26. The method of claim 18, wherein after administration of the vector, the method further comprises:
inducing expression of the functional Nav1.1 sodium channel.

27. The method of claim 18, wherein the vector is administered to an individual with a missense mutation or a nonsense mutation in SCNIA, or to an individual with a truncation mutation in SCN1A, or to an individual with a mutation that affects splicing of SCN1A transcripts, or to an individual with a frameshift mutation involving SCN1A, or to an individual with a mutation in a promoter/enhancer/regulatory region of SCN1 A.

28-31. (canceled)

32. The method of claim 18, wherein after administration of the vector, the method further comprises:
monitoring the mammal for desired clinical results, and if the desired clinical results are not obtained;
administering a second therapeutically effective amount of the vector.

33. (canceled)

34. The method of claim 18, further comprising:
administering a therapeutically effective amount of seizure medication, or
administering a therapeutically effective amount of Alzheimer's disease medication, or
administering a therapeutically effective amount of epilepsy medication.

35-36. (canceled)

37. The method of claim 18, wherein:
a therapeutically effective amount of the vector is administered for the treatment of Dravet Syndrome, or
a therapeutically effective amount of the vector is administered for the treatment of epilepsy, or
a therapeutically effective amount of the vector is administered for the treatment of Alzheimer's disease, or
a therapeutically effective amount of the vector is administered for the treatment of autism spectrum disorder, or
a therapeutically effective amount of the vector is administered for the treatment of neurodevelopmental disorders.

38-41. (canceled)

42. A composition for providing exogenous Nav1.1 channels, the composition comprising:
the vector of claim 1.
